(19) United States
(12) Patent Application Publication
(10) Pub. No.: US 2025/0092192 A1
(43) Pub. Date: Mar. 20, 2025

(54) IONIZABLE POLYESTERS, POLYPLEXES AND METHODS OF USE

(71) Applicant: University of Maryland, Baltimore, Baltimore, MD (US)

(72) Inventors: Ryan M. Pearson, Baltimore, MD (US); Atanu Chakraborty, Baltimore, MD (US)

(21) Appl. No.: 18/832,457

(22) PCT Filed: Feb. 1, 2023
(86) PCT No.: PCT/US2023/012123

§ 371 Date: Jul. 23, 2024

Related U.S. Application Data
(60) Provisional application No. 63/305,308, filed on Feb. 1, 2022.

(51) Int. Cl. C08G 63/685; A61K 47/34; C08G 63/81; C08G 63/90

(52) U.S. Cl. CPC C08G 63/6854; A61K 47/34; C08G 63/81; C08G 63/90; C08G 2230/00

(57) ABSTRACT

The present invention provides ionizable polyesters, compositions comprising ionizable polyester polyplexes, and methods of delivering nucleic acids to cells comprising administering the polyplexes to subjects.

### CROSS REFERENCE TO RELATED APPLICATIONS

This application claims the benefit of U.S. Provisional Appl. No. 63/305,308, filed Feb. 1, 2022, the contents of which are hereby incorporated by reference in their entirety.

### FIELD OF THE INVENTION

The field of the invention relates generally to compositions and methods for delivering nucleic acids to cells.

### BACKGROUND

Nucleic acid delivery to immune cells has the potential to revolutionize therapeutic outcomes for various diseases. However, the delivery of nucleic acids to primary immune cells, particularly macrophages, presents several challenges that limit transfection efficiency. First, macrophages contain degradative enzymes that destroy nucleic acids after transfection and limit expression of exogenous genes (Odaka et al., Role of Macrophage Lysosomal Enzymes in the Degradation of Nucleosomes of Apoptotic Cells., (1999), 163:5346-5352). Second, the low-proliferative nature of primary cells restricts the entry of extracellular DNA molecules into the nucleus, which leads to marginal transfection (Maury et al., Biomaterials (2014), 35:5977-85). Viral and non-viral vectors have been developed to enhance nucleic acid delivery. Viral vectors (adenoviruses, adeno-associated viruses, lentiviruses) are highly efficient and have been used successfully for several preclinical and clinical applications⁴ but these vectors present potential safety concerns including mutagenesis, immunogenicity, and cytotoxicity (Lee et al., Genes & Diseases, (2017), 4:43-63; Colella et al., Mol Ther Methods Clin Dev, (2018), 8:87-104; Shirley et al., Mol Ther, (2020), 28:709-722; Hacein-Bey-Abina, et al., (2010), 363:355-364; van den Berg et al., Journal of Controlled Release, (2021), 331:121-141). Non-viral gene delivery methods, on the other hand, offer the ability to overcome many of these limitations, while enabling a wide chemical space to be accessed to develop libraries of materials to investigate structure-function relationships though tuning of composition, surface functionality, and other physicochemical properties (van den Berg et al., Journal of Controlled Release, (2021), 331:121-141; Hong, et al., Sugar-based gene delivery systems: current knowledge and new perspectives, (2018), 181:1180-1193; Arote et al., Degradable poly (amido amine) s as gene delivery carriers, (2011), 8:1237-1246; Hong et al., Sugar alcohol-based polymeric gene carriers: synthesis, properties and gene therapy applications, (2019), 97:105-115). Recently, significant advancements have been made in the design of non-viral nucleic acid delivery platforms including lipid nanoparticles (LNPs) and polymer-based systems (Lungwitz et al., European journal of pharmaceutics and biopharmaceutics: official journal of Arbeitsgemeinschaft fur Pharmazeutische Verfahrenstechnik e.V, (2005), 60:247-66; Toncheva, Biochimica et Biophysica Acta (BBA)-General Subjects, (1998), 1380:354-368; Wang et al., Polyamidoamine Dendrimer Grafted with an Acid-Responsive Charge-Reversal Layer for Improved Gene Delivery, (2020), 21:4008-4016; Karlsson et al., Expert Opinion on Drug Delivery, (2020), 17:1395-1410; Gao, X., In Gene Therapy for Diseases of the Lung, CRC Press: (2020), pp 99-112; Mohammadinejad et al., Journal of Controlled Release, (2020), 325:249-275; Liu et al., European Journal of Medicinal Chemistry, (2017), 129:1-11; Loh et al., Biomaterials Science, (2016), 4:70-86; Hou et al., Nature Reviews Materials, (2021), 6:1078-1094). LNPs have received significant interest due to their remarkable success as a delivery platform for COVID-19 mRNA vaccines (Hou et al., Nature Reviews Materials, (2021), 6:1078-1094). These LNPs consist of a mixture of multiple components and excipients including ionizable lipid, helper lipid, PEG-lipid, and cholesterol to enable adequate qualities for mRNA delivery. Despite the advantages of LNPs, lipid-based molecules are less stable and difficult to synthesize and purify, thus limiting their cost-effectiveness (Mitchell et al., Nature Reviews Drug Discovery, (2021), 20:101-124). To facilitate the wide implementation of nucleic acid-based therapeutics, identification of alternative non-viral delivery platforms with reduced complexity that do not compromise delivery efficacy is required. Off the shelf cationic polymers such as polyethylenimine (PEI), poly(l-lysine) (PLL), and others have been thoroughly examined for nucleic acid delivery applications; yet their in vitro efficacy often does not effectively translate to in vivo applications. Strategies such as increasing the amine to phosphate (N:P) ratio offers the possibility to improve the transfection abilities of these platforms but compromises their toxicity profile (Chakraborty et al., ACS Applied Bio Materials, (2020), 3:6263-6272). Functional polyester-based carriers containing ionizable subunits are advantageous due to their biodegradability, modularity, adaptability to high throughput synthesis, and low toxicity (Piperno et al., Int J Nanomedicine, (2021), 16:5981-6002). For example, poly(β-amino ester) s (PBAE) are a common platform used for plasmid DNA (pDNA) and mRNA transfection; however, successful delivery to immune cells typically requires functional modification of the polymer backbone or the terminal groups in addition to the incorporation of excipients such as lipids or other polymers for surface coating to enable efficient transfection (Rui et al., Science Advances, (2022), 8: eabk2855; Kaczmarek, et al., Nano Letters, (2018), 18:6449-6454; Kim et al., Advanced drug delivery reviews, (2021), 170:83-112; Zhang et al., Nature Communications, (2019), 10:3974).

There is a need for new compounds and compositions capable of delivering nucleic acids to cells in vivo.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

Described herein is a one-pot synthesis of hydrophobic ionizable polyesters as a single-component nucleic acid delivery platform.

In some aspects, the ionizable polyesters have high serum and enzymatic stability and are capable of efficient transfection of “hard-to-transfect” innate immune cells with lung-selective mRNA transfection in vivo. To identify the impact of polymer design parameters on transfection, a set of ionizable polyesters (polyMDET and polyMDET-Cp) was synthesized via a polycondensation reaction from commercially-available N-methyldiethanolamine (MDET), four alkyl diols (p=4, 6, 8, 10), and sebacoyl chloride. Polyplex stability was determined and transfection efficiency using immortalized RAW 264.7 cells and primary macrophages derived from bone marrow (BMDM) was assessed in vitro without the incorporation of any excipients or targeting ligands. Systemic administration of polyplexes by intravenous injection showed over 23-fold lung-selective protein expression and efficient transfection of lung alveolar macrophages and dendritic cells with no detectable organ toxicity or systemic inflammation.

In one aspect, the present invention provides an ionizable polyester having the following structure:

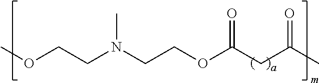

wherein a is an integer in a range of from 5 to 12; and wherein m is in a range of from about 11 to about 190 (polyMDET-ADA). Note that ADA is an acronym for alkanedioic acid.

In important embodiments, the ionizable polyester has the following structure:

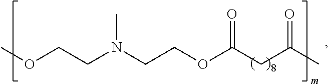

wherein m is in a range of from about 14 to about 160 (polyMDET). In some embodiments, the polyMDET polyester can be prepared by condensation of N-methyldiethanolamine (MDET) and sebacoyl chloride, wherein the condensation can be performed as a one pot synthesis.

In another aspect, the present invention provides an ionizable polyester having the following structure:

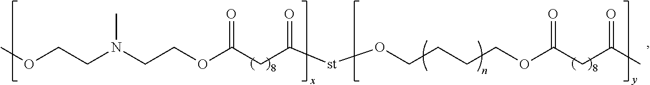

wherein n is 1, 2, 3, 4, 5 or 6; wherein x is in a range of from about 10 to about 130;
wherein y is in a range of from about 3 to about 40; and wherein “st” signifies that the polyester includes a statistical distribution of the two bracketed primary units (polyMDET-Cp).

In embodiments, this terpolymer polyester can be prepared by condensation of N-methyldiethanolamine (MDET), sebacoyl chloride and a 1, p-alkanediol, wherein p is [(n*2)+2], and wherein the condensation is performed as a one pot synthesis.

In certain embodiments, a hydrophobicity of the polyester increases with increasing n.

In certain embodiments, the y to x ratio can be in a range of from about 0.25 to about 0.35.

In useful embodiments, the above described polyesters can have high serum and enzymatic stability in a mammalian body.

In some embodiments, the polyester can be biodegradable.

In some embodiments, the polyester can exhibit low toxicity to mammals.

In some embodiments, the molecular weight of the polyester can be in a range of from about 4000 daltons to about 4500 daltons.

In another aspect, the present invention provides a method of preparing poly[(N-methyl diethanolamine)-alt-(alkanedioic acid)], comprising providing q moles of N-methyldiethanolamine (MDET) dissolved in a first nonaqueous solvent, mixing 2q moles of a proton scavenger with the MDET, stirring under an inert atmosphere for about 15 minutes, adding q moles of sebacoyl chloride dissolved in a second nonaqueous solvent to the mixture of MDET with the proton scavenger over about 30 minutes to provide a reaction mixture, continuing to stir the reaction mixture for 12 hours under an inert atmosphere, forming a solid hydrochloride salt of the proton scavenger during the adding and continuing steps, removing the solid hydrochloride salt from the reaction mixture; and evaporating the solvent(s) to provide a crude polyester product.

In some embodiments, the method of preparing poly[(N-methyl diethanolamine)-alt-(alkanedioic acid)] polyester can further comprise dissolving the crude polyester product in a minimum amount of methanol to form a methanol solution, precipitating a purified polyester product by adding the methanol solution to diethyl ether, isolating the purified polyester product by filtration and drying the purified polyester product for two days at room temperature.

In some embodiments of the method of preparing poly[(N-methyl diethanolamine)-alt-(alkanedioic acid)] polyester, the first nonaqueous solvent and the second nonaqueous solvent can be dichloromethane. In some embodiments, the proton scavenger can be pyridine. In some embodiments, the inert atmosphere can be argon. In some embodiments, the removing step can be accomplished by centrifugation. In some embodiments, the evaporating step can be accomplished with a rotary evaporator. In some embodiments, the concentration of MDET in the first nonaqueous solvent in the providing step can be about 1.0M. In some embodiments, the concentration of sebacoyl chloride in the second nonaqueous solvent in the adding step can be about 1.0M.

In another aspect, the present invention provides a method of preparing poly[((N-methyl diethanolamine)-alt-(sebacic acid))-stat-((1, p-alkanediol)-alt-(sebacic acid))] polyester, comprising providing q moles of N-methyldiethanolamine (MDET) and (0.2) q moles of 1,p-alkanediol, wherein p is [2n+2], mixed and dissolved in a first nonaqueous solvent, mixing (2.4) q moles of a proton scavenger with the MDET and 1,p-alkanediol, stirring under an inert atmosphere for about 15 minutes, adding (1.2) q moles of sebacoyl chloride dissolved in a second nonaqueous solvent to the mixture of MDET with the proton scavenger over about 30 minutes to provide a reaction mixture, continuing to stir the reaction mixture for 12 hours under an inert atmosphere, forming a solid hydrochloride salt of the proton scavenger during the adding and continuing steps, removing the solid hydrochloride salt from the reaction mixture and evaporating the solvent(s) to provide a crude polyester product.

In some embodiments, the method of preparing poly[((N-methyl diethanolamine)-alt-(sebacic acid))-stat-((1,p-alkanediol)-alt-(sebacic acid))] polyester, where p is 4, 6, 8, or 10 can further comprise dissolving the crude polyester product in a minimum amount of methanol to form a methanol solution, precipitating a purified polyester product by adding the methanol solution to diethyl ether, isolating the purified polyester product by filtration and drying the purified polyester product for two days at room temperature.

In some embodiments of the method of preparing poly[((N-methyl diethanolamine)-alt-(sebacic acid))-stat-((1,p-alkanediol)-alt-(sebacic acid))] polyester, the first nonaqueous solvent and the second nonaqueous solvent can be dichloromethane. In some embodiments, the proton scavenger can be pyridine. In some embodiments, the inert atmosphere can be argon. In some embodiments, the removing step can be accomplished by centrifugation. In some embodiments, the evaporating step can be accomplished with a rotary evaporator. In some embodiments, the concentration of MDET in the first nonaqueous solvent in the providing step can be about 1.0M. In some embodiments, the concentration of sebacoyl chloride in the second nonaqueous solvent in the adding step can be about 1.0M.

In another aspect, the present invention provides a stable polyplex comprising an effective amount of one of the above described polyesters and either an effective amount of plasmid DNA (pDNA) or an effective amount of messenger RNA (mRNA).

In some embodiments, the polyplex was formed from the polyester and either pDNA or mRNA at a pH of 5.

In some embodiments, the polyplex can have a size of from about 100 nm to about 300 nm at a pH of 5. In other embodiments, the polyplex can have a size of from about 300 nm to about 600 nm at a pH of 7.4. In some embodiments, the same polyplex can exhibit both of these characteristics. In some embodiments, the polyplex can have a larger size at a pH of 7.4 as compared with its size at a pH of 5.

In some embodiments, the polyplex can have a positive zeta potential of at least about 40 mV at a pH of 5. In other embodiments, the polyplex having a negative zeta potential of less than about-20 mV at a pH of 7.4.

In some embodiments, the polyplex can be formed from the polyester and pDNA, wherein the polyplex comprises a polyester to pDNA weight ratio of from about 30:1 to about 260:1. In some embodiments, the polyplex can comprise a polyester to pDNA weight ratio of from about 55:1 to about 220:1. In particular embodiments, the polyplex can comprise a polyester to pDNA weight ratio of 55:1, 110:1, or 220:1.

In some embodiments, the polyplex can be formed from the polyester and mRNA, wherein the polyplex comprises a polyester to mRNA weight ratio of from about 30:1 to about 80:1.

In some embodiments, the polyplex can comprise a polyester to pDNA or mRNA weight ratio of about 55:1.

In some embodiments, wherein the polyester is polyMDET-ADA or polyMDET, the polyplex can have a size of from about 500 nm to about 600 nm at a pH of 7.4.

In some embodiments, wherein the polyester is polyMDET-Cp, the polyplex can have a size of from about 300 nm to about 400 nm at a pH of 7.4.

In some embodiments, wherein the polyplex is any of the above described polyplexes, the pDNA or mRNA in the polyplex can be protected from degradation by human serum enzymes.

In some embodiments, wherein the inventive polyplex was formed from the polyester and pDNA, the pDNA in the polyplex can be protected from degradation by DNase I enzyme.

In another aspect, the present invention provides a method of delivering a nucleic acid to a target organ, comprising administering an effective amount of any of the above described polyplexes to a mammalian subject. In some embodiments, the subject is a human. In other embodiments, the subject is a mouse.

In some embodiments, the administration can be via intravenous injection.

In some embodiments, transfection of the pDNA or mRNA of the polyplex into cells of the subject can be accomplished.

In some embodiments, lung and spleen cells can be transfected in strong preference to cells of any other organ. In some embodiments, lung and spleen cells can be transfected in strong preference to cells of the liver, heart and kidneys. In some embodiments, lung and spleen cells can be transfected in at least a 12-fold preference to cells of the liver, heart and kidneys.

In some embodiments, lung cells can be transfected in strong preference to cells of any other organ. In some embodiments, lung cells can be transfected in strong preference to cells of the liver, heart and kidneys.

In some embodiments of the inventive method of delivering a nucleic acid to a target organ, innate immune cells can be effectively targeted. In some embodiments, the targeted innate immune cells can be macrophages.

In some embodiments of the inventive method, no excipient to the polyplex is required in order to deliver the nucleic acid to the target organ.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. Synthesis and characterization of ionizable polyesters. Synthetic schemes for (A) polyMDET and (B) polyMDET-Cp polyesters, respectively. The polyesters were synthesized via a polycondensation reaction between N-methyldiethanolamine (MDET) and sebacoyl chloride for polyMDET or using MDET, sebacoyl chloride, and various alkyl diols for polyMDET-Cp, where “p” stands for the total number of carbons in the aliphatic chains. (C) Representative ¹H NMR spectra of polyMDET and polyMDET-C6. The peak at 4.2 ppm and 1.2-1.5 ppm signifies the protons for MDET and sebacoyl chloride. The peak around 4 ppm (8) indicates the protons of the hexyl group, demonstrating successful incorporation into the polymer backbone. (D) Molecular weight and quantification of MDET and Cp determined by MALDI-TOF MS and ¹H NMR spectra.

FIG. 2. Polyplex preparation and characterization. (A) Schematic representation of the polyplex preparation with polymers and pDNA or mRNA. (B) Agarose gel electrophoresis showing the stability of polyplexes made with various polymers and pDNA at different weight ratios (55:1, 110:1, and 220:1). (C) Agarose gel electrophoresis showing the stability of polyplexes prepared using various polymers and mRNA at 55:1 weight ratio. Hydrodynamic size and zeta potential of polyplexes prepared with (D) pDNA and (E) mRNA, respectively. Data are representative of n=3 experiments. Errors bars represent standard deviation. Left arrow indicates the hydrodynamic diameter of the polyplexes, whereas the right arrow indicates the zeta potential.

FIG. 3. Transfection of GFP encoded pDNA and mRNA in RAW 264.7 cells. (A) RAW 264.7 cells were transfected with polyMDET/pDNA or polyMDET-Cp/pDNA and polyMDET/mRNA or polyMDET-Cp/mRNA polyplexes prepared at a weight ratio of 110:1. GFP was used to assess for the transfection efficiency of the polyplexes. Scale bar 100 μm. (B) Representative gating strategy for the quantification of transfection by flow cytometry. Flow cytometry-based quantification of transfection efficiency of (C) polyMDET/pDNA and polyMDET-Cp/pDNA and (E) polyMDET/mRNA, polyMDET-Cp/mRNA polyplexes, respectively. Mean fluorescence intensity (MFI) of GFP expression after transfection of (D) GFP-encoded pDNA and (F) mRNA, respectively. PEI₃₀ corresponds to polyplexes prepared at N/P ratio 30. The amount of GFP pDNA and GFP mRNA used was 2 μg and 1 μg, respectively per 2×10⁵ RAW 264.7 cells. Data are representative of n=3 experiments. Statistical differences between groups were determined by performing a one-way ANOVA and Tukey's post-hoc test (** p<0.01, **** p<0.0001). Errors bars represent standard deviation.

FIG. 4. Flow cytometry-based quantification of transfection efficiency of polyMDET and polyMDET-Cp polyplexes in primary BMDM cells. (A) Representative schematic for extraction of bone marrow from the femur and tibia of C57BL/6 mouse, differentiation into bone marrow-derived macrophages (BMDM), and polyplex treatment. Created with BioRender. (B and D) Flow cytometry-based quantification of transfection efficiency of polyMDET/pDNA, polyMDET-Cp/pDNA and polyMDET/mRNA, polyMDET/mRNA polyplexes, respectively, in BMDMs. (C and E) Mean fluorescence intensity (MFI) of GFP expression after transfection of GFP encoded pDNA and mRNA, respectively. The amount of GFP pDNA and GFP mRNA used was 2 μg and 1 μg, respectively per 2×10⁵ BMDMs. PEI30 corresponds to polyplexes prepared at N/P ratio 30. Data are representative of n=3 experiments. Statistical differences between groups were determined by performing a one-way ANOVA and Tukey's post-hoc test (*** P<0.001 **** P<0.0001). Errors bars represent standard deviation.

FIG. 5. Uptake mechanism study of polyMDET/pDNA and polyMDET-Cp/pDNA polyplexes in RAW 264.7 cells. RAW 264.7 macrophages were incubated with various endocytosis inhibitors for 1 hour before treatment with the polyplexes. The MFI of GFP expression was analyzed by flow cytometry after 24 hours. The amount of GFP pDNA used was 2 μg per 2×10⁵ RAW 264.7 cells. Data are representative of n=3 experiments. Statistical differences between groups were determined by performing a one-way ANOVA and Tukey's post-hoc test (*P<0.05, ** P<0.01, *** P<0.001 **** P<0.0001). Errors bars represent standard deviation.

FIG. 6. In vivo mRNA transfection in C57BL/6 mice and quantification of immune cell transfection. (A) Schematic for mRNA polyplex preparation, in vivo administration, and analysis of cell transfection. (B) Representative ex vivo bioluminescence images of organs after 24 h of intravenously injected FLuc-encoded mRNA polyplexes in C57BL/6 mice. (C) Quantification of FLuc mRNA expression in selected tissues. (D) Relative bioluminescence intensity of lung compared to spleen in PolyMDET, PolyMDET-C4, and PolyMDET-C6. Flow cytometric analysis in the lung of (E) total percentage of CD45⁺GFP⁺ cells. (F) total percentage of CD45⁻GFP⁺cells. (G) total percentage of CD45⁺F4/80⁻CD11c⁺GFP⁺. (H) total percentage of CD45⁺F4/80⁺CD11c⁻GFP⁺ (I) CD45⁺F4/80⁺CD11c⁺cells in lungs. Statistical differences between groups were determined by performing a one-way ANOVA and Turkey's post-hoc test (*P<0.05, ** P<0.01, *** P<0.001 **** P<0.0001). Errors bars represent standard deviation. N=3 mice per experimental group.

FIG. 7. In vivo histological examination and proinflammatory cytokine levels of polyplex-treated mice. (A) Histopathological changes of various organ tissues after single dose intravenous administration of polyplexes (10 μg mRNA) in mice. Scale bar 200 μm. (B) Expression of plasma proinflammatory cytokines after intravenous administration of the polyplexes using ELISA. mRNA polyplexes did not show any proinflammatory cytokine IL-6 and TNFa expression after 24 h. Lipopolysaccharide (LPS) used as positive control induced higher amount of IL-6 and TNFa expression. nd—not detected. N=3 mice per experimental group.

FIG. 8. ¹H NMR characterization of ionizable polyesters. ¹H NMR spectra are calibrated to 2.5 ppm using DMSO-d6 or 7.2 ppm using CDCl₃.

FIG. 9. 500 MHz 2D DOSY NMR spectra obtained at 298 K in DMSO-d6 solution of the polymers. The diffusion coefficients (as shown through the DOSY maps) were not distinctly different for the signals detected within each polymer sample confirming successful polymerization and negligible levels of monomers or oligomers present.

FIG. 10. MALDI-TOF spectra of the polyMDET and polyMDET-Cp polymers. The mass difference between two main peaks is 285, which corresponds to MDET blocks.

FIG. 11. Gel electrophoresis of polyMDET-C6/pDNA at various weight ratios. The polyplexes were able to fully condense pDNA at a 55:1 (Polymer: pDNA) ratio, however incomplete complexation was observed at lower ratios.

FIG. 12. Determination of encapsulation efficiency of pDNA and mRNA. The standard curve was obtained by measuring the emission intensity of DAPI and RiboGreen following incubation with free pDNA or mRNA respectively. The polyplexes were prepared as described in the experimental section and following centrifugation, the amount of unencapsulated pDNA or mRNA was measured using a fluorescence plate reader using DAPI or RiboGreen. The encapsulation efficiency as shown in the table was measured by subtracting the total amount of pDNA or mRNA used and free pDNA or mRNA left in the supernatant. Data are representative of n=3 experiments.

FIG. 13. Buffering capacity of the polymers. Titration of acidified polymers with 0.1 N NaOH. pKa was determined from the pH of the half neutralization value for each polymer. pKa for polyMDET=5.01, polyMDET-C4=4.17, polyMDET-C6=4.9, polyMDET-C8=4.2.

FIG. 14. The variation of hydrodynamic diameter over 7 days. Polyplexes were prepared and stored at pH 5 at 4° C. Prior to each size measurement, the polyplexes were dialyzed against DPBS buffer (pH 7.4). Data are representative of n=3 experiments. Errors bars represent standard deviation.

FIG. 15. Stability of the polyplexes on day 1 and day 7. Polymers formed stable polyplexes after mixing with pDNA, however partial release of pDNA was observed after 1 week.

FIG. 16. Determination of the stability of the encapsulated pDNA in serum. The polyplexes were incubated with 55% of FBS (physiological concentration) for 1 hour before loading into the gel. The clear band at the top of the well shows that the pDNA is stably complexed within polyplexes.

FIG. 17. Gel electrophoresis of the polyplexes after incubation with DNase I enzyme. Free pDNA was degraded by the enzyme after 1 hour incubation as there is no band observed, whereas the polyplexes can resist the degradation of pDNA.

FIG. 18. Cytotoxicity assessment of the polyplexes. Cell viability was determined by the MTS assay in RAW 264.7 cells. Polyplexes prepared with polymers and pDNA (weight ratio of polymers and pDNA 110:1) were incubated with RAW 264.7 cells for 4 hours, washed, and incubated for 24 hours prior to analysis. Data are representative of n=3 experiments. Statistical differences between groups were determined by performing a one-way ANOVA and Tukey's post-hoc test (p<0.05). Errors bars represent standard deviation.

FIG. 19. Histograms representing the mean fluorescence intensity of GFP expression following transfection of pDNA and mRNA in (A) RAW 264.7 cells and (B) primary BMDM cells.

FIG. 20. Fluorescence microscopy imaging of GFP mRNA transfection in BMDM cells. The polyplexes were prepared by mixing various ionizable polyesters and GFP mRNA at 110:1 weight ratio and incubated with BMDM cells for 3 hours. The images showed that polyMDET-C6 is more efficient to transfect BMDMs.

FIG. 21. Endosomal escape of the polymer particles. Cy5.5 dye was encapsulated in the polyMDET-C6 and incubated with RAW 264.7 cells for 3 h. Cells were then washed and incubated for 24 h with fresh media and imaged using a fluorescence microscope. The arrows show areas where the particles are not co-localized with lysosomes.

FIG. 22. Biodistribution study of Cy5.5-encapsulated FLuc mRNA polyplexes. The IVIS® images show the distribution of polyplexes in various organs and their corresponding organ transfection.

FIG. 23. Representative gating scheme used for flow cytometry analysis of GFP⁺ expression in lung cell populations. DAPI was used to distinguish live and dead cells. PerCP-CD45 (clone 30-F11) PE-Cy7 F4/80 (clone BM8) and BV605 anti-mouse CD11c (clone N418) were used to distinguish various immune cells such as CD45⁺GFP⁺ lymphocytes, CD45⁻GFP⁺non-lymphocytes. CD45⁺F4/80⁻CD11c⁺GFP⁺ (dendritic cells), CD45⁺F4/80⁺CD11c⁻GFP⁺ (interstitial macrophages) and CD45⁺F4/80⁺CD11c⁺ (alveolar macrophages). Quadrant gates were drawn based on PBS injected control mice for defining GFP⁺ cells.

FIG. 24. Flow cytometric analysis of in vivo CD11c⁺ and F4/80⁻ cell transfection in the spleen. Data are representative of n=3 experiments. Statistical differences between groups were determined by performing a one-way ANOVA and Tukey's post-hoc test (*P<0.05, ** P<0.01, *** P<0.001 **** P<0.0001). Errors bars represent standard deviation. N=3 mice per experimental group.

FIG. 25. Flow cytometric analysis of in vivo immune cell (except macrophages and dendritic cells) transfection in spleen and lung. Data shows that there is no significant difference in transfection of F4/80⁻CD11c⁻ immune cell populations but around 1.5-2.5% of F4/80⁻CD11c⁻ immune cells are transfected in lungs by GFP mRNA polyMDET-C4 and polyMDET-C6 polyplexes. Statistical differences between groups were determined by performing a one-way ANOVA and Tukey's post-hoc test (*P<0.05, *** P<0.001). Errors bars represent standard deviation. N=3 mice per experimental group.

FIG. 26. PBS Lung.

### DETAILED DESCRIPTION

Nucleic acid-based therapeutics have great potential to alter the future of medical intervention. In particular, polymer-based nucleic acid delivery systems have been developed for extrahepatic nucleic acid delivery, but complexities associated with the synthesis and purification of custom monomers, post-synthetic modifications, and incorporation of additional excipients to improve their stability and endosomal escape qualities pose challenges to their clinical translation. Described herein is a one pot synthetic method for preparing novel ionizable polyesters from N-methyldiethanolamine (MDET) and sebacoyl chloride such that these monomers can alternate along the polyester chain, with optional addition to the pot of 1,p-alkanediols having a saturated linear hydrocarbon chain p carbon atoms in length with hydroxy groups on either end, such that a terpolymer forms in a statistical manner. These ionizable polyesters can be used to form polyplex associations with nucleic acids such as pDNA or mRNA. The resulting polyplex nanoparticles display high serum and enzymatic stability and effectively deliver pDNA or mRNA to “hard-to-transfect” primary macrophages in vitro when administered by intravenous injection. The present inventors found that the described polyplex nanoparticles can deliver genetic materials to immune cells of the lungs in strong preference to cells of other mammalian organs and with high levels of protein expression. The result is a simple, excipient-free, non-viral nucleic acid delivery platform with lung-selective and innate immune cell tropism that has potential to expedite the progress of polymer-based genetic medicines into the clinic.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

In some embodiments, the practice of the present invention employs various techniques of molecular biology (including recombinant techniques), microbiology, cell biology, chemistry, biochemistry and immunology. See, e.g., Sambrook et al. Molecular Cloning: A Laboratory Manual, 2nd edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); Animal Cell Culture (R. I. Freshney ed. (1987)); and Handbook of Thermoplastic Polymers: Homopolymers, Copolymers, Blends, and Composites (Fakirov, ed. (2002), Wiley-VCH Verlag GmbH & Co. KGaA); Kopnick, et al., “Polyesters,” in Ullmann's Encyclopedia of Industrial Chemistry (Wiley-VCH Verlag GmbH & Co. KGaA, 2000).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “an antibody” includes a plurality of antibodies, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

The terms “effective amount” or “therapeutically effective amount” or “therapeutic effect” refer to an amount of an agent described herein, an antibody, a polypeptide, a polynucleotide, an amine containing cationic biomaterial, an anionic biomaterial, a small organic molecule, or other drug effective to deliver nucleic acids to cells, or otherwise “prevent” or “treat” a disease or disorder in a subject such as, a mammal.

The term “nanoparticle” as used herein refers to a particle having a size from about 1 nm to about 1000 nm.

The terms “nucleic acid,” and “polynucleotide,” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties.

The term “particle” as used herein refers to a composition having a size from about 1 nm to about 1000 μm.

The term “particle size” as used herein refers to the median size in a distribution of particles. The median size is determined from the average linear dimension of individual particles, for example, the diameter of a spherical particle. Size may be determined by any number of methods in the art, including dynamic light scattering (DLS) and transmission electron microscopy (TEM) techniques.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids. Such polypeptides can be wild-type or a variant, modified or chimeric polypeptide. A “variant polypeptide” can mean a modified polypeptide such that the modified polypeptide has an amino acid alteration compared to wild-type polypeptide.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented.

The term “subject” refers to any animal (e.g., a mammal), including, but not limited to, humans, non-human primates, canines, felines, rabbits, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of an agent described herein, an antibody, a polypeptide, a polynucleotide, an amine containing cationic biomaterial, an anionic biomaterial, a small organic molecule, or other drug effective to deliver nucleic acids to cells, or otherwise “prevent” or “treat” a disease or disorder in a subject such as, a mammal.

In some embodiments, the invention provides an ionizable polyester having the following structure:

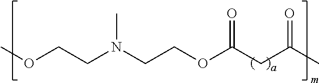

wherein a is an integer in a range of from 5 to 12; and
wherein m is in a range of from about 11 to about 190.

In some embodiments, the ionizable polyester has the following structure:

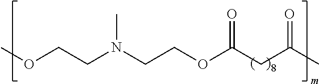

wherein m is in a range of from about 14 to about 160.

In some embodiments, the ionizable polyester is prepared by condensation of N-methyldiethanolamine (MDET) and sebacoyl chloride.

In some embodiments, the invention provides an ionizable polyester having the following structure:

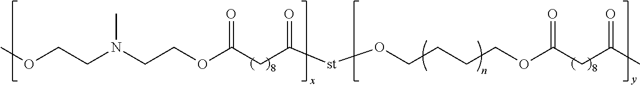

wherein n is 1, 2, 3, 4, 5 or 6;
wherein x is in a range of from about 10 to about 130;
wherein y is in a range of from about 3 to about 40; and
   wherein “st” signifies that the polyester includes a statistical distribution of the two bracketed primary units.

In some embodiments, the ionizable polyester is prepared by condensation of N-methyldiethanolamine (MDET), sebacoyl chloride and a 1,p-alkanediol, wherein p is [(n*2)+2], and wherein the condensation is performed as a one pot synthesis.

In some embodiments, the hydrophobicity of the polyester increases with increasing n.

In some embodiments, the y to x ratio is in a range of from about 0.25 to about 0.35. In some embodiments, the polyester has high serum and enzymatic stability when administered to a subject.

In some embodiments, the polyester is biodegradable.

In some embodiments, the polyester exhibits low toxicity when administered to a subject.

The ionizable polyesters can be prepared by any suitable method.

For polyMDET-ADA polyesters including polyMDET, preparative methods known in the art include reaction of N-methyldiethanolamine (MDET) with a saturated linear alkanedioyl chloride, reaction of MDET with poly(sebacic anhydride), reaction of MDET with sebacic acid, transesterification by reaction of MDET with dimethyl sebacate, for example, and reaction of MDET with decanedinitrile. Jalal, et al., New limited molecular weight polymeric dispersants prepared by melt condensation polymerization, Chemistry and Materials Research 2014, 6 (2): 12-18. The polyesters can be formed by, for example, step-growth polycondensation, ring-opening polymerization, polyaddition reactions, or melt condensation polymerization. These reactions can be conducted with or without the presence of a catalyst.

In certain embodiments, the polyMDET-Cp polyesters can be prepared by any of the above methods by adding an effective amount of 1,p-alkanediol to the MDET, where p is the number of carbon atoms in the saturated linear hydrocarbon diol. The reactions can still be performed in a single pot, the result being a statistical occurrence of either MDET or the alkanediol, alternating in the polyester polymer chain with sebacic acid monomer. According to the present inventors' proton NMR integrations of the polyesters, the usual result is a molar amount of alkanediol incorporated into the polyester of about 30% of the molar amount of MDET incorporated into the polyester.

A convenient laboratory synthesis of the subject polyesters can be performed by reaction of a difunctional acid chloride such as sebacoyl chloride with MDET, the MDET optionally being mixed with a 1,p-alkanediol having a linear hydrocarbon chain of p carbon atoms. The reaction is non-reversible and efficient when an acid scavenger such as a tertiary amine or pyridine is used. The reaction can typically be run at room temperature.

Polycondensation of alkanedioyl chlorides with hydrocarbon diols might not be suitable for large scale production because of the high cost and corrosiveness of the alkanedioyl chlorides, as well as their sensitivity to hydrolysis and other side reactions resulting from the high reactivity of the acid chlorides. The byproduct amine salts might represent a disposal problem or require separate recycling as well. Other methods such as transesterification or direct esterification typically require temperatures in excess of 200C in order to get good conversion. The latter reactions are usually reversible, however, and can be moved forward by removal of byproducts such as water or methanol. Synthesis of linear saturated polyesters, in Polymer Properties Database, Crow, 2015-2022, polymerdatabase.com (accessed Jan. 25, 2023). Barot, et al., Polyester the workhorse of polymers: a review from synthesis to recycling, Arch. Appl. Sci. Res. 2019, 11 (2): 19; Baharu, et al., Synthesis and characterization of polyesters derived from glycerol, azelaic acid, and succinic acid, Green Chemistry Letters and Reviews 2015, 8 (1): 31-38.

Skilled practitioners will appreciate that sebacic acid can be derived from renewable biomass resources and that polyesters derived from this and other bio-based monomers have been widely studied in recent years due to their “tunable properties and wide range of application areas.” Zhang, et al., Bio-based polyesters: recent progress and future prospects, Progress in Polymer Science 2021, 120:101430; Zia, et al., Recent developments and future prospects on bio-based polyesters derived from renewable resources: a review, International Journal of Biological Macromolecules 2016, 82:1028-1040. Tuning is accomplished through control of a combination of monomer structure and molecular weight. Some sebacic acid-based polymers have FDA approval for biomedical applications, and some have suggested that adjusting viscoelastic properties could lead to polymers suitable for use in cell delivery systems or soft tissue augmentation. Park, et al., Synthesis of elastic biodegradable polyesters of ethylene glycol and butylene glycol from sebacic acid, Acta Biomaterialia 2012, 8 (8): 2911-2918; Brannigan, et al., Synthesis, properties and biomedical applications of hydrolytically degradable materials based on aliphatic polyesters and polycarbonates, Biomater. Sci. 2016, 5 (1): 9-21. More broadly, functional aliphatic polyesters are known for their versatility in biomedical applications such as protein delivery and scaffolds for tissue engineering, offering many possibilities for tuning characteristics such as hydrophobicity, degradation rate, extent of crystallinity and glass transition temperature. Seyednejad, et al., Functional aliphatic polyesters for biomedical and pharmaceutical applications, Journal of Controlled Release 2011, 152 (1): 168-176. Thus, in the context of the present invention, optimization according to known methods of polyester properties that relate to nucleic acid delivery is contemplated to be within the scope of the present invention. Such optimization can include a variety of derivatives and minor backbone modifications of the polyesters described herein. Variations in molecular weight of the polyesters are also contemplated.

In particular, the present inventors contemplate that variations in the length of the saturated hydrocarbon chain of the sebacic acid monomer can be made in order to adjust hydrophobicity. Hydrophobicity of the polyester can affect transfection efficiency. Specifically, the present invention can comprise a poly[(N-methyl diethanolamine)-alt-(alkanedioic acid)] polyester, where alkanedioic acid is selected from the group consisting of pimelic acid, suberic acid, azelaic acid, sebacic acid, undecanedioic acid, dodecanedioic acid, tridecanedioic acid and tetradecanedioic acid. More particularly, the polyester of the present invention can be poly[(N-methyl diethanolamine)-alt-(sebacic acid)]. Other ways of modifying hydrophobicity can include derivatizing the saturated hydrocarbon chain of the alkanedioic acid monomer with one or more substituents including but not limited to methyl, ethyl, propyl, n-butyl, sec-butyl, tert-butyl, hydroxy, methoxy, ethoxy, propoxy and acetyloxy.

Another way to modify hydrophobicity in the polyester is to add an alkanediol as a third monomer to be incorporated in the one pot synthesis of the subject polyesters. Specifically, the present invention can comprise a poly[((N-methyl diethanolamine)-alt-(sebacic acid))-stat-((1,p-alkanediol)-alt-(sebacic acid))] polyester, where p is an integer in a range of from 3 to 10, and where alkanediol is a saturated linear alkyl hydrocarbon chain having p carbon atoms and being derivatized at either end with a hydroxyl group. More particularly, p can be selected from the group consisting of 4, 6, 8 and 10. In this series, hydrophobicity of the polyester increases with increasing p.

Molecular weights of the polyesters of the present invention are not necessarily limiting and can be in a range of from about 2000 daltons to about 7000 daltons. More particularly, molecular weights of the polyesters of the present invention can be in a range of from about 4000 daltons to about 4500 daltons. For example, the molecular weight of a polyester of the present invention can be about 4500, about 4450, about 4400, about 4350, about 4300, about 4250, about 4200, about 4150, about 4100, about 4050, or about 4000 daltons.

Molecular weights and their distribution can be determined using various methods known in the art, including size exclusion chromatography and gel permeation chromatography.

Variations and permutations of the described methods for preparing the inventive polyesters are considered to be within the scope of the present invention. Various aprotic nonpolar solvents are suitable, including benzene, diethyl ether, dioxane, 1,2-dimethoxyethane, chororform, etc., as well as dichloromethane. The inert atmosphere can be achieved with inert gases such as nitrogen, as well as argon. Various proton scavengers such as tertiary amines (e.g., triethylamine) or pyridine or its derivatives can be used. Acyl chlorides are highly reactive, so even very short reaction times can be expected to produce some polyester product. The inert atmosphere is primarily necessary in order to prevent hydrolysis of the acyl chloride by ambient moisture, but some polyester yield would be produced without this precaution. Other methods of purifying the crude polyester products can be developed, such as using different solvent combinations to obtain a purified product. Gel permeation chromatography can also be used.

### Polyplex Particles and Methods of Use

Previously, polyplexes based on low molecular weight polymers were generally found to exhibit lower colloidal stability as compared with higher molecular weight polymers having comparable structure, and low molecular weight polymers had a reputation for marginal transfection properties. Luten, et al., Biodegradable polymers as non-viral carriers for plasmid DNA delivery, Journal of Controlled Release 2008, 126 (2): 97-110. Polyesters having molecular weights of less that about 10,000 daltons are generally considered to be of relatively low molecular weight. The present invention overcomes these difficulties.

In embodiments, the present invention provides a stable polyplex comprising an effective amount of the any of the above described ionizable polyesters and an effective amount of nucleic acid, such as, for example, plasmid DNA (pDNA) or messenger RNA (mRNA).

The nucleic acid to be transfected is not particularly limited. In some embodiments, the nucleic acid is selected from the group consisting of DNA, messenger RNA, small interfering RNA (siRNA), short hairpin RNA (shRNA), antisense oligonucleotides (ODN) and combination thereof. In some embodiments, the nucleic acid is selected from the group consisting of an antisense oligonucleotide, cDNA, genomic DNA, guide RNA, plasmid DNA, vector DNA, mRNA, miRNA, piRNA, shRNA, lncRNA, siRNA, and combinations thereof. In some embodiments, the nucleic acid is not siRNA. In some embodiments, the DNA comprises plasmid DNA. As used herein, the term “plasmid DNA” refers to a small DNA molecule that is typically circular and is capable of replicating independently.

In some embodiments, the nucleic acid encodes a fragment of a polypeptide. In some embodiments, the nucleic acid comprises a template nucleic acid that can be useful, e.g., to modify the genome of the cell. In some embodiments, the nucleic acids encode one or more nucleases useful for modifying the genome of the cell.

In some embodiments, the nucleic acid encodes or modulates the expression of one or more proteins. Non-limiting examples include a blood clotting factor (e.g., Factor XIII, Factor IX, Factor X, Factor VIII, Factor VIIa, or protein C), apoE2, TPP1, argininosuccinate synthase, copper transporting ATPase 2, acid alpha-glucosidase, β-Glucocerebrosidase, α-galactosidase, C1 inhibitor serine protease inhibitor, CFTR (cystic fibrosis transmembrane regulator protein), an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor-3 and -4, brain-derived neurotrophic factor, glial derived growth factor, transforming growth factor .alpha. and .beta., etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g., that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes, or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, gene deficiencies causative of lysosomal storage diseases (e.g., sulfatases, N-acetylglucosamine-1-phosphate transferase, cathepsin A, GM2-AP, NPC1, VPC2, Sphingolipid activator proteins, etc.), one or more zinc finger nucleases for genome editing, and/or donor sequences used as repair templates for genome editing.

In some embodiments, the nucleic acid encodes or modulates the expression of one or more cytokines. In some embodiments, the nucleic acid reduces expression of an endogenous cytokine. In some embodiments, the cytokine is selected from the group consisting of transforming growth factor-beta (TGF-beta), interferons (e.g., interferon-alpha, interferon-beta, interferon-gamma), colony stimulating factors (e.g., granulocyte colony stimulating factor (GM-CSF)), thymic stromal lymphopoietin (TSLP), and the interleukins, e.g., interleukin-1, interleukin-2, interleukin-3, interleukin-4, interleukin-5, interleukin-6, interleukin-7, interleukin-8, interleukin-10, interleukin-12, interleukin-13, interleukin-15, interleukin-17, interleukin-18, interleukin-22, interleukin-23, and interleukin-35.

In some embodiments, the nucleic acid encodes a therapeutic antibody or an Fc fusion protein useful in the treatment of an inflammatory disease. Anti-inflammatory antibodies include adalimumab, alemtuzumab, atlizumab, canakinumab, certolizumab, certolizumab pegol, daclizumab, efalizumab, fontolizumab, golimumab, infliximab, mepolizumab, natalizumab, omalizumab, ruplizumab, ustekinumab, visilizumab, zanolimumab, vedolizumab, belimumab, otelixizumab, teplizumab, rituximab, ofatumumab, ocrelizumab, epratuzumab, eculizumab, and briakinumab. Exemplary useful Fc fusion proteins to treat inflammatory diseases include atacicept, abatacept, alefacept, etanercept, and rilonacept.

In some embodiments, the nucleic acid modulates expression of one or more polypeptide hormones. In some embodiments, the nucleic acid encodes the polypeptide hormone. In some embodiments, the nucleic acid reduces expression of an endogenous polypeptide hormone. In some embodiments, the polypeptide hormone is selected from the group consisting of amylin, anti-Mullerian hormone, calcitonin, cholecystokinin, corticotropin, endothelin, enkephalin, erythropoietin (EPO), follicle-stimulating hormone, gallanin, gastrin, ghrelin, glucagon, gonadotropin-releasing hormone, growth hormone-releasing hormone, hepcidin, human chorionic gonadotropin, human growth hormone (hGH), inhibin, insulin, insulin-like growth factor, leptin, luteinizing hormone, luteinizing hormone releasing hormone, melanocyte stimulating hormone, motilin, orexin, oxytocin, pancreatic polypeptide, parathyroid hormone, prolactin, secretin, somatostatin, thrombopoietin, thyroid-stimulating hormone, vasoactive intestinal peptide, and vasopressin.

In some embodiments, the nucleic acid (e.g., siRNA, miR, mRNA) is capable of modulating the expression and/or activity of one or more proteins (e.g., an enzyme, e.g., a kinase) associated with a cancer. In some embodiments, the nucleic acid can target a protein selected from the group consisting of: kinesin spindle protein (KSP), RRM2, keratin 6a (K6a), HER1, ErbB2, a vascular endothelial growth factor (VEGF) (e.g., VEGFR1, VEGFR3), a platelet-derived growth factor receptor (PDGFR) (e.g., PDGFR-α, PDGFR-β), epidermal growth factor receptor (EGFR), a fibroblast growth factor receptor (FGFR) (e.g., FGFR1, FGFR2, FGFR3, FGFR4), EphA2, EphA3, EphA4, HER2, HER3, HER4, INS-R, IGF-1R, IR-R, CSF1R, KIT, FLK-II, KDR/FLK-1, FLK-4, flt-1, c-Met, Ron, Sea, TRKA, TRKB, TRKC, FLT3, VEGFR/Flt2, Flt4, EphA1, EphB2. EphB4, Pim1, Pim2, Pim3, Tie2PKN3, PLK1, PLK2, PLK3, Src, Fyn, Lck, Fgr, Btk, Fak, SYK, FRK, JAK, Abl, Kit, KDR, CaM-kinase, phosphorylase kinase, MEKK, ERK, mitogen activated protein (MAP) kinase, phosphatidylinositol-3-kinase (PI3K), an AKT (e.g., Akt1, Akt2, Akt3), TGF-βR, KRAS, BRAF, a cyclin-dependent kinase (e.g., CDK1, CDK2, CDK4, CDK5, CDK6, CDK7, and CDK9), GSK3, a CDC-like kinase (CLK) (e.g., CLK1, CLK4), an Aurora kinase (e.g., Aurora A, Aurora B, and Aurora C), a mitogen-activated protein kinase (MEK) (e.g., MEK1, MEK2), mTOR, protein kinase A (PKA), protein kinase C (PKC), protein kinase G (PKG), and PHB1.

In some embodiments, the nucleic acid encodes an antibody molecule. In some embodiments, the nucleic acid encodes a therapeutic antibody molecule capable of treating cancer. In some embodiments, the nucleic acid sequence encodes variable domain sequences of an antibody selected from abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, and zalutumumab.

In some embodiments, the nucleic acid encodes a chimeric antigen receptor (CAR). In some embodiments, the CAR may include an antigen binding domain, a transmembrane domain, a costimulatory signaling region, and a CD3 zeta signaling domain. In some embodiments, the antigen binding domain may bind to an antigen of a non-essential organ. The CAR can be expressed in a target cell selected from the group consisting of a T cell, a natural killer (NK) cell, NK-92 cell, a cytotoxic T lymphocyte (CTL), and a regulatory T cell.

CARs are molecules generally including an extracellular and intracellular domain. The extracellular domain includes a target-specific binding element. The intracellular domain (e.g., cytoplasmic domain) includes a costimulatory signaling region and a zeta chain portion. The costimulatory signaling region refers to a portion of the CAR including the intracellular domain of a costimulatory molecule. Costimulatory molecules are cell surface molecules other than antigens receptors or their ligands that are required for an efficient response of lymphocytes to antigen. Between the extracellular domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain of the polypeptide chain. A spacer domain may include up to 300 amino acids, preferably 10 to 100 amino acids, and most preferably 25 to 50 amino acids.

In some embodiments, the target-specific binding element of the CAR in the present disclosure may recognize a tumor antigen. Tumor antigens are proteins that are produced by tumor cells that elicit an immune response, particularly T-cell mediated immune responses. Tumor antigens are well known in the art and include, for example, a glioma-associated antigen, carcinoembryonic antigen (CEA), β-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxylesterase, mut hsp70-2, M-CSF, prostase, prostate-specific antigen (PSA), PAP, NY-ESO-1, LAGE-la, p53, prostein, PSMA, Her2/neu, survivin and telomerase, prostate carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor .alpha. 2. IL-11 receptor .alpha., MUC1, MUC16, CA9, GD2, GD3, HMW-MAA, CD171, Lewis Y, G250/CAIX, HLA-A1 MAGE A1, HLA-A2 NY-ESO-1, PSC1, folate receptor-.alpha., CD44v7/8, 8H9, NCAM, VEGF receptors, 514, Fetal AchR, NKG2D ligands, CD44v6, TEM1, TEM8, or viral-associated antigens expressed by the tumor.

In some embodiments, the binding element of the CAR may include any antigen binding moiety that when bound to its cognate antigen, affects a tumor cell such that the tumor cell fails to grow, or is promoted to die or diminish.

In some embodiments, the nucleic acid encodes or is an RNA interfering agent. As used herein, an “RNA interfering agent” is defined as any agent that interferes with or inhibits expression of a target gene, e.g., by RNA interference (RNAi). Such RNA interfering agents include, but are not limited to, antisense molecules, ribozymes, small inhibitory nucleic acid sequences, for example but not limited to guide RNAs, small interfering RNA (siRNA), short hairpin RNA or small hairpin RNA (shRNA), microRNA (miRNA), post-transcriptional gene silencing RNA (ptgsRNA), short interfering oligonucleotides, antisense oligonucleotides, aptamers, CRISPR RNAs, nucleic acid molecules including RNA molecules which are homologous to the target gene, or a fragment thereof, and any molecule which interferes with or inhibits expression of a target gene by RNA interference (RNAi).

Non-limiting examples of target genes where it is desirable to inhibit expression include huntingtin (HTT) gene, a gene associated with dentatorubropallidolusyan atrophy (e.g., atrophin 1, ATN1); androgen receptor on the X chromosome in spinobulbar muscular atrophy, human Ataxin-1, -2, -3, and -7, Cav2.1 P/Q voltage-dependent calcium channel is encoded by the (CACNA1A), TATA-binding protein, Ataxin 8 opposite strand, also known as ATXN8OS, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DAN damage-inducible transcript 4 protein, in diabetic macular oedma (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection (e.g., spike protein in SARS-COV-2); respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; mutant rhodopsin gene (RHO) in autosomal dominantly inherited retinitis pigmentosa (adRP); or the inhibitory nucleic acid binds to a transcript of any of the foregoing genes or sequences.

In some embodiments, the polyplex particle further comprises a targeting moiety that enables delivery of the nucleic acids to a target cell, wherein the targeting moiety binds to the surface of the target cell, wherein the non-viral polyplex particle is internalized by the target cell by receptor mediated endocytosis.

In some embodiments, the targeting moiety is selected from the group consisting of a protein, a cell adhesion molecule, an antibody, a peptide, a sugar, a small molecule, and any combination thereof.

In some embodiments, the targeting moiety comprises a single chain antibody.

In some embodiments, the targeting moiety comprises a single chain (scFv) variable fragment antibody.

In some embodiments, the polyplex particle comprises a targeting moiety that binds to a cell surface molecule or complex on an immune cell. In some embodiments, the targeting moiety binds to a cell surface molecule or complex selected from the group consisting of mannose receptor (CD206), folic acid receptor, Ly6G, Ly6C and CD3. The targeting moieties could be various types of targeting ligands such as mannose, folic acid, tuftsin peptide, anti-Thy1.1. In some embodiments the targeting moiety is an anti-CD3e F(ab)₂ molecule. In some embodiments, the targeting moiety is conjugated to the anionic biomaterial, thereby enabling presentation of the targeting moiety at the outer surface of the polyplex particle. In some embodiments, the targeting moiety is conjugated to poly(ethylene-alt-maleic anhydride).

In some embodiments, the polyplex particle comprises a targeting moiety that binds to a cell surface molecule or complex on a cancer cell.

The term “N/P ratio” refers to the molar ratio of nitrogen (N) in the ionizable polyester to the total phosphate (P) in nucleic acid. In some embodiments, the polyplex particle has an N/P ratio of at least 7.5. In some embodiments, the polyplex particle has an N/P ratio of at least 15. In some embodiments, the polyplex particle has an N/P ratio of at least 30.

In some embodiments, the N/P ratio of the polyplex particles can be optimized to transfect specific cell types, such as immune cells. In some embodiments, polyplex particles have an N/P ratio of at least 15 for transfecting macrophages. In some embodiments, polyplex particles have an N/P ratio of at least 7.5 for transfecting T cells. In some embodiments, polyplex particles have an N/P ratio of about 30. In some embodiments, the cells to be transfected are selected from lung cells, spleen cells, and immune cells.

In some embodiments, the polyplex was formed from the polyester and either pDNA or mRNA at a pH of 5, where the subject polyesters can be expected to be cationic. Polyplexes can generally be prepared in a mixed solvent comprising an aqueous buffer and a water miscible organic solvent that effectively dissolves the polyester. An aqueous acetate buffer is particularly suitable. However, other inert and water soluble materials having a pKa near 5 can be used to form the buffer. Examples are phthalate, citrate, succinate, pyridine and hexamethylenetetramine. In an exemplary procedure, polyester solutions in dimethyl sulfoxide (DMSO) (55 mg/mL) in an amount of 2.0 μL, 3.6 μL, or 7.3 μL were added to 50 μL portions of aqueous acetate buffer (25 mM, pH=5). A quantity of 2 μg of nucleic acid was then added; this provides polyester to nucleic acid weight ratios of 55:1, 100:1 and 200:1, respectively. Incubation at room temperature provides the polyplexes. Variations in this procedure, such as use of a different organic solvent, a different reagent concentration, or a different organic solvent/water ratio, can be used. However, using a minimum amount of organic solvent necessary to maintain solubility of the polyester is desirable in order to minimize distortion of measurement of the actual pH of the mixed solvent solution.

In some embodiments, the particle size of the polyplex particle can be in a range from about 10 nm to about 10 mm. In some embodiments, the size can be in a range from about 10 nm to about 1000 nm, from about 100 nm to about 950 nm, from about 150 nm to about 800 nm, and/or from about 200 nm to about 600 nm. For example, in some embodiments, the particle size can be about 100 nm, about 125 nm, about 150, about 175 nm, about 200 nm, about 2250 nm, about 250 nm, about 275 nm, about 300 nm, about 325 nm, about 350 nm, about 375 nm, about 400 nm, about 425 nm, about 450 nm, about 475 nm, about 500 nm, about 550 nm, about 600 nm, about 650 nm, about 700 nm, about 750 nm, about 800 nm, about 850 nm, about 900 nm, about 950 nm, or about 1000 nm.

In some embodiments, the polyplex particles present within a population, e.g., in a composition, can have substantially the same shape and/or size (i.e., they are “monodisperse”). For example, the particles can have a distribution such that no more than about 5% or about 10% of the particles have a diameter greater than about 10% greater than the average diameter of the particles, and in some cases, such that no more than about 8%, about 5%, about 3%, about 1%, about 0.3%, about 0.1%, about 0.03%, or about 0.01% have a diameter greater than about 10% greater than the average diameter of the particles.

In some embodiments, the diameter of no more than 25% of the particle varies from the mean particle diameter by more than 150%, 100%, 75%, 50%, 25%, 20%, 10%, or 5% of the mean particle diameter. It is often desirable to produce a population of particle that is relatively uniform in terms of size, shape, and/or composition so that most of the particles have similar properties. For example, at least 80%, at least 90%, or at least 95% of the particles produced using the methods described herein can have a diameter or greatest dimension that falls within 5%, 10%, or 20% of the average diameter or greatest dimension. In some embodiments, a population of particles can be heterogeneous with respect to size, shape, and/or composition.

In some embodiments, the polyplex can have a size of from about 100 nm to about 300 nm at a pH of 5. For example, the polyplex size at a pH of 5 can be about 100, about 125, about 150, about 175, about 200, about 225, about 250, about 275, or about 300 nm. In other embodiments, the polyplex can have a size of from about 300 nm to about 600 nm at a pH of 7.4. For example, the polyplex size at a pH of 7.4 can be about 300, about 325, about 350, about 375, about 400, about 425, about 450, about 475, about 500, about 525, about 550, about 575, or about 600 nm. In some embodiments, the same polyplex can exhibit both of these characteristics. In some embodiments, the polyplex can have a larger size at a pH of 7.4 as compared with its size at a pH of 5.

In some embodiments, the polyplex can have a positive zeta potential of at least about 40 mV at a pH of 5. In other embodiments, the polyplex can have a negative zeta potential of less than about-20 mV at a pH of 7.4.

In some embodiments, the polyplex can be formed from the polyester and pDNA, wherein the polyplex comprises a polyester to pDNA weight ratio of from about 30:1 to about 260:1. In some embodiments, the polyplex can comprise a polyester to pDNA weight ratio of from about 55:1 to about 220:1. In particular embodiments, the polyplex can comprise a polyester to pDNA weight ratio of 55:1, 110:1, or 220:1.

More particularly, the weight ratio of polyester to pDNA in the inventive polyplex can be about 30:1, about 40:1, about 50:1, about 60:1, about 70:1, about 80:1, about 90:1, about 100:1, about 110:1, about 120:1, about 130:1, about 140:1, about 150:1, about 160:1, about 170:1, about 180:1, about 190:1, about 200:1, about 210:1, about 220:1, about 230:1, about 240:1, about 250:1, or about 260:1.

In some embodiments, the polyplex can be formed from the polyester and mRNA, wherein the polyplex comprises a polyester to mRNA weight ratio of from about 30:1 to about 80:1.

More particularly, the weight ratio of polyester to mRNA in the inventive polyplex can be about 30:1, about 40:1, about 50:1, about 60:1, about 70:1, or about 80:1.

In some embodiments, the polyplex can comprise a polyester to pDNA or mRNA weight ratio of about 55:1.

In some embodiments, wherein the polyester is polyMDET-ADA or polyMDET, the polyplex can have a size of from about 500 nm to about 600 nm at a pH of 7.4. More particularly, these polyplexes can have a size of about 500, about 510, about 520, about 530, about 540, about 550, about 560, about 570, about 580, about 590, or about 600 nm.

In some embodiments, wherein the polyester is polyMDET-Cp, the polyplex can have a size of from about 300 nm to about 400 nm at a pH of 7.4. More particularly, these polyplexes can have a size of about 300, about 310, about 320, about 330, about 340, about 350, about 360, about 370, about 380, about 390, or about 400 nm.

In some embodiments, wherein the polyplex is any of the above described polyplexes, the pDNA or mRNA in the polyplex can be protected from being degraded by human serum enzymes.

In some embodiments, wherein the inventive polyplex was formed from the polyester and pDNA, the pDNA in the polyplex can be protected from being degraded by DNase I enzyme.

In another embodiment, the invention provides a method of delivering a nucleic acid to a target cell, comprising administering an effective amount of the polyplex particles as described herein.

In another embodiment, the invention provides a method of delivering a nucleic acid to a target cell in a subject, comprising administering an effective amount of the polyplex particles as described herein. Advantageously, administration of the polyplex nanoparticles to a mammalian subject can be by intravenous injection.

The methods can be performed in vitro, in vivo or ex vivo. When performed in vitro or ex vivo, the methods can be performed in the presence or absence of serum. Advantageously, the polyplex particles can be efficiently delivered and taken up by cells in vivo, in the presence of serum.

In some embodiments, the invention provides for a method of treating a disease or condition in a subject in need thereof, comprising administering to the subject a therapeutically effective amount of a composition as described herein.

The compositions of the disclosure can be used in any method of treating a disease or condition beneficially treated by administration of a nucleic acid in a subject.

The cell that can be delivered the nucleic acid by the polyplex particle is not particularly limiting. The cell becomes transfected upon uptake of the polyplex particle. The term “transfect” refers to introduction of a molecule such as a nucleic acid (plasmid) into a cell. A cell has been “transfected” when exogenous nucleic acid has been introduced inside the cell membrane. Accordingly, a “transfected cell” is a cell into which a “nucleic acid” or “polynucleotide” has been introduced, or a progeny thereof in which an exogenous nucleic acid has been introduced. In particular embodiments, a “transfected cell” cell (e.g., in a mammal, such as a cell or tissue or organ cell) is a genetic change in a cell following incorporation of an exogenous molecule, for example, a nucleic acid (e.g., a transgene). A “transfected” cell(s) can be propagated and the introduced nucleic acid transcribed and/or protein expressed.

In a “transfected” cell, the nucleic acid (plasmid) may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extrachromosomally, or only transiently.

In some embodiments, the transfected cell is selected from a blood cell, a cancer cell, an immune cell (e.g., a macrophage cell), an epithelial cell (e.g., a skin cell), or a virus-infected cell.

In some embodiments, immune cells are transfected. In some embodiments, the immune cell is selected from neutrophils, bone marrow cells, bone marrow stem cells, NK cells, dendritic cells, monocytes, B lymphocytes, macrophages, and T lymphocytes.

In some embodiments, the transfected cell is a cancer cell. In some embodiments, the cancer cell is selected from a breast cancer cell, a colon cancer cell, a leukemia cell, a bone cancer cell, a lung cancer cell, a bladder cancer cell, a brain cancer cell, a bronchial cancer cell, a cervical cancer cell, a colorectal cancer cell, an endometrial cancer cell, an ependymoma cancer cell, a retinoblastoma cancer cell, a gallbladder cancer cell, a gastric cancer cell, a gastrointestinal cancer cell, a glioma cancer cell, a head and neck cancer cell, a heart cancer cell, a liver cancer cell, a pancreatic cancer cell, a melanoma cancer cell, a kidney cancer cell, a laryngeal cancer cell, a lip or oral cancer cell, a lymphoma cancer cell, a mesothioma cancer cell, a mouth cancer cell, a myeloma cancer cell, a nasopharyngeal cancer cell, a neuroblastoma cancer cell, an oropharyngeal cancer cell, an ovarian cancer cell, a thyroid cancer cell, a penile cancer cell, a pituitary cancer cell, a prostate cancer cell, a rectal cancer cell, a renal cancer cell, a salivary gland cancer cell, a sarcoma cancer cell, a skin cancer cell, a stomach cancer cell, a testicular cancer cell, a throat cancer cell, a uterine cancer cell, a vaginal cancer cell, and a vulvar cancer cell. For example, the cancer cell can be a lung cancer cell.

The polyplex particles and methods of the invention can be used to treat any number of diseases in a subject, including cancer. Cancers include, but are not limited to, an adrenal cancer, a breast cancer, a colon cancer, a leukemia, a bile duct cancer, a bone cancer, a lung cancer (e.g., non-small cell lung cancer, small cell lung cancer, and lung carcinoid tumor), a bladder cancer, a brain cancer, a bronchial cancer, a cervical cancer, a colorectal cancer, an endometrial cancer, an ependymoma, a retinoblastoma, a gallbladder cancer, a gastric cancer, a gastrointestinal cancer, a glioma, a head and neck cancer, a heart cancer, a liver cancer, a pancreatic cancer, a melanoma, a kidney cancer, a laryngeal cancer, a lip or oral cancer, a lymphoma, a mesothioma, a mouth cancer, a myeloma, a nasopharyngeal cancer, a neuroblastoma, an oropharyngeal cancer, an ovarian cancer, a thyroid cancer, a penile cancer, a pituitary cancer, a prostate cancer, a rectal cancer, a renal cancer, a salivary gland cancer, a sarcoma, a skin cancer, a stomach cancer, a testicular cancer, a throat cancer, a uterine cancer, a vaginal cancer, and a vulvar cancer.

In some embodiments, the methods of the present invention can be used to treat an inflammatory disease or disorder, which can include sepsis, arthritis, multiple sclerosis, rheumatoid arthritis, psoriasis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis, Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, polymyalgia rheumatica, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, reperfusion injury, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis. Inflammatory diseases or conditions with an inflammatory component not triggered by autoimmunity are also included. See, for example, Tabas, I; Glass, C. K. Science 339 (6116): page 169 (2013).

In some embodiments, the methods enable reprogramming of a dysregulated immune response in a subject, for example, to improve cell migration, overcome significant cell death induced by over stimulation, increase pathogen clearance, or reduce apoptosis induction in T cells. In some embodiments, the methods deliver siRNA specifically to T cells targeting pro-apoptotic Bcl-2 family member Bim and Fas during T cell apoptosis in sepsis.

In some embodiments, the methods of the invention can be used to treat viral or bacterial infections. In some embodiments, the methods of the invention can be used to prevent or to treat a viral disease in a subject. In some embodiments, the viral disease is selected from the group consisting of: Adenovirus infections, Herpes virus infections (e.g., HSV-1, HSV-2 and varicella zooster virus infections), Papillomavirus infections (e.g., HPV-1, HPV-2, HPV-5, HPV-6, HPV-11, HPV-13, HPV-16, and HPV-18), Parvovirus infections, Polyomavirus infections, Poxvirus infections, Arbovirus infection, Arenavirus infections, Astrovirus infections, Birnavirus infections, Bunyavirus infections, Calicivirus infections, Coronavirus infections (e.g., SARS-COV-2), Flavivirus infections, Hantavirus infections, Hepatitis virus infections (e.g., Hepatitis A, Hepatitis B, Hepatitis C, and Hepatitis D), Bornavirus infections, Filovirus infections, (e.g., Ebola virus, Marburg virus, and Cueva virus), Paramyxovirus infections (e.g., respiratory syncytial virus), and Rhabdovirus infections), Nidovirales infections, Orthomyxoviridae infections (e.g., influenza virus infections), Picornavirus infections (e.g., Enterovirus infections), Reovirus infections (e.g., Rotavirus infections), Retrovirus infections (e.g., lentivirus infections, e.g., HIV infections), and Togavirus infections (e.g., Rubivirus infections). In some embodiments, the nucleic acid (e.g., siRNA) prevents the replication of a virus causing disease. In some embodiments, the polyplex particles can function as vaccines by delivering nucleic acids encoding antigens to antigen presenting cells, such as dendritic cells. In some embodiments, the polyplex particle comprises a targeting moiety that targets uptake of the nucleic acid by antigen presenting cells, such as dendritic cells. In some embodiments, the nucleic acid encodes a polypeptide comprising a viral antigen or antigenic fragment.

In some embodiments, the methods of the invention can be used to treat a disease or a condition in need of enzyme (i.e., gene) replacement in a subject. For example, MPS disorders (mucopolysaccharidoses) are lysosomal storage diseases caused by the inability to produce specific enzymes, which in turn leads to an abnormal storage of mucopolysaccharides. In some embodiments, the disease in need of enzyme replacement is selected from the group consisting of: Gaucher disease, Fabry disease, Hurler syndrome (MPS I H), Scheie syndrome (MPS I S), Hurler-Scheie syndrome (MPS I H-S), Hunter syndrome (MPS II), Sanfilippo syndrome (e.g. Sanfilippo A (MPS III A), Sanfilippo B (MPS III B), Sanfilippo C (MPS III C), and Sanfilippo D (MPS III D)), Morquio syndrome (e.g. Morquio A (MPS IV A) and Morquio B (MPS IV B)), Maroteaux-Lamy syndrome (MPS VI), Sly syndrome (MPS VII), MPS IX (hyaluronidase deficiency), I-cell disease (ML II), Pseudo-Hurler polydystrophy (ML III), and Glycogen storage disease type II (Pompe disease). In some embodiments, the nucleic acid encodes a polypeptide useful to treat a disease in need of enzyme replacement. In some embodiments, the nucleic acid encodes a protein (e.g., an enzyme) that is deficient and/or less active in a subject suffering from a disease in need of enzyme replacement. In some embodiments, the protein comprises one or more proteins selected from the group consisting of: agalsidase beta, imiglucerase, velaglucerase alfa, taliglucerase, alglucosidase alfa, laronidase, idursulfase, and galsulfase.

### Pharmaceutical Compositions

When employed as pharmaceuticals, the polyplex particles of the invention can be administered in the form of pharmaceutical compositions.

One of the advantages of the present invention is that it can deliver nucleic acid to cells without use of the excipients commonly employed in lipid nanoparticle delivery of nucleic acids. In some embodiments, the pharmaceutical compositions herein can achieve transfection of nucleic acid, such as pDNA or mRNA, preferentially into lung or spleen cells.

Excipients that can optionally be used in certain embodiments of the present invention, but which are not required, can include lipid nanoparticles, helper lipids, poly(ethylene glycol) lipids and cholesterol. Helper lipids can include, for example, 1,2-dioleoyl-sn-glycero-3-polyethylene (DOPE), or the O-pegylated derivative of the N,N-dimyristylamide of 2-hydroxyacetic acid (ALC-0159), available from BroadPharm, San Diego, CA.

Thus the present disclosure provides a composition comprising a polyplex particle, or a pharmaceutically acceptable salt thereof, or any of the embodiments thereof, and at least one pharmaceutically acceptable carrier. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject and which does not destroy the pharmacological activity of the polyplex particle and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

This invention also includes pharmaceutical compositions which contain, as the active ingredient, the polyplex particle of the invention or a pharmaceutically acceptable salt thereof, in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active particle, soft and hard gelatin capsules, suppositories, sterile injectable solutions and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the subject by employing procedures known in the art.

In some embodiments, the pharmaceutical composition comprises silicified microcrystalline cellulose (SMCC) and at least one polyplex particle described herein, or a pharmaceutically acceptable salt thereof. In some embodiments, the silicified microcrystalline cellulose comprises about 98% microcrystalline cellulose and about 2% silicon dioxide w/w.

In some embodiments, the compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

In some embodiments, the active polyplex particle may be effective over a wide dosage range and is generally administered in a therapeutically effective amount. It will be understood, however, that the amount of the particle actually administered will usually be determined by a physician, according to the relevant circumstances, including the condition to be treated, the chosen route of administration, the actual particle administered, the age, weight, and response of the individual subject, the severity of the subject's symptoms and the like.

In some embodiments, the therapeutic dosage of a polyplex particle of the present invention can vary according to, e.g., the particular use for which the treatment is made, the manner of administration of the particle, the health and condition of the subject, and the judgment of the prescribing physician. The proportion or concentration of a particle of the invention in a pharmaceutical composition can vary depending upon a number of factors including dosage, chemical characteristics (e.g., hydrophobicity), and the route of administration. For example, the particles of the invention can be provided in an aqueous physiological buffer solution containing about 0.1 to about 10% w/v of the particle for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular subject, the relative biological efficacy of the particle selected, formulation of the excipient, and its route of administration. Effective doses can be extrapolated from dose-response curves derived from in vitro or animal model test systems.

In some embodiments, the liquid forms in which the polyplex particles and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tert, or intermittent positive pressure breathing machine. Solution, suspension, or powder compositions can be administered orally or nasally from devices which deliver the formulation in an appropriate manner.

In some embodiments, topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the polyplex particles of the invention. The topical formulations can be suitably packaged in tubes of, e.g., 100 g which are optionally associated with instructions for the treatment of the select indication, e.g., psoriasis or other skin condition.

In some embodiments, the amount of polyplex particle or composition administered to a subject will vary depending upon what is being administered, the purpose of the administration, such as prophylaxis or therapy, the state of the subject, the manner of administration and the like. In therapeutic applications, compositions can be administered to a subject already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the subject and the like.

In some embodiments, the compositions administered to a subject can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the particle preparations typically will be between 3 and 11, more preferably from 5 to 9 and most preferably from 7 to 8. It will be understood that use of certain of the foregoing excipients, carriers or stabilizers will result in the formation of pharmaceutical salts.

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

The present invention is further illustrated by the following Examples. These Examples are provided to aid in the understanding of the invention and are not to be construed as a limitation thereof.

### Examples

### Example 1. Excipient-free Ionizable Polyester Nanoparticles for Lung-selective and Innate Immune Cell Plasmid DNA and mRNA Transfection

Design and Synthesis of polyMDET and polyMDET-Cp

Biodegradable polymers can overcome several challenges associated with non-viral gene delivery, including in vivo stability, susceptibility to enzymatic degradation, poor cellular uptake, and inefficient endosomal escape. Ionizable polyesters as DNA/RNA delivery platforms containing tertiary amines and synthesized from custom monomers have gained attention due to their abilities to facilitate a pH-dependent, charge-altering characteristic based on the pKa of the polymer. Further, post-synthetic modifications of side chains or end groups allow for tunable hydrophobicity or alternative charge characteristics to be engineered for optimal nucleic acid delivery.

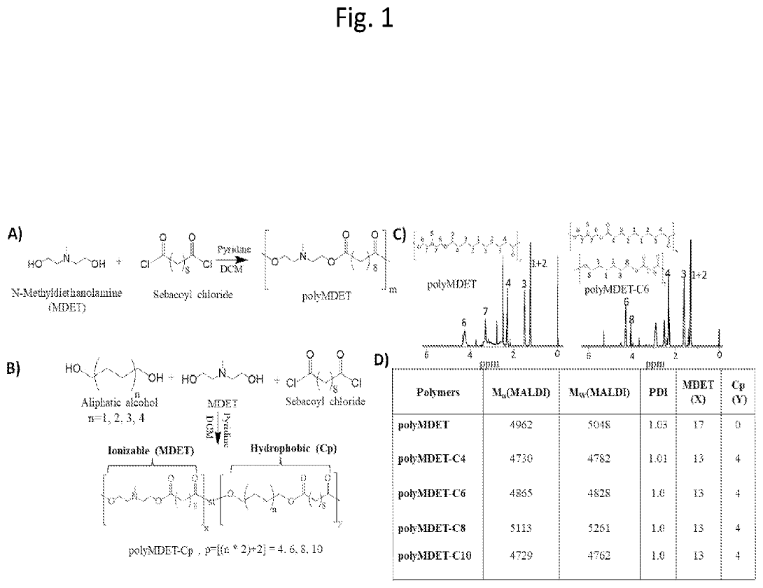

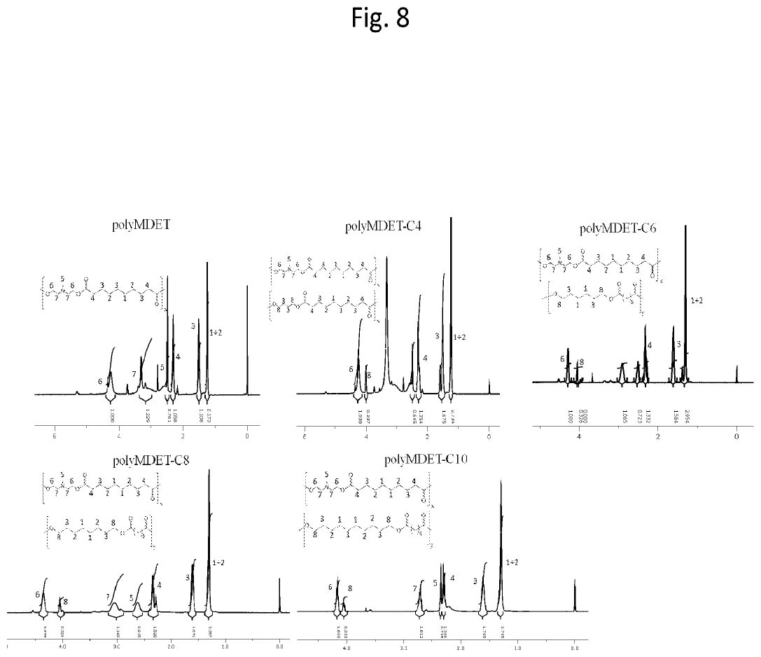

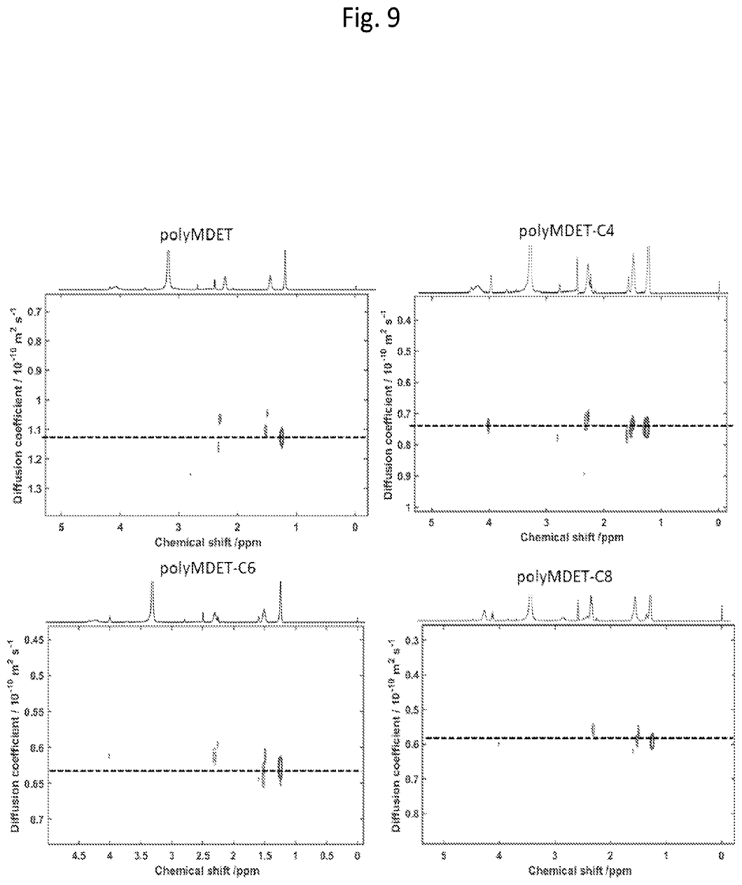

Recognizing the need for less complex non-viral nucleic acid delivery platforms, we hypothesized that we could utilize commercially-available monomers in a one-pot reaction to create a single-component nucleic acid delivery platform for efficient pDNA and mRNA transfection in vitro and in vivo. We synthesized a set of ionizable polyesters containing tertiary amines and various hydrophobic alkyl diols by condensation polymerization (FIGS. 1A and B). The main components of the polymers were MDET and sebacoyl chloride (e.g. polyMDET) and alkyl diols that varied in chain length (e.g. polyMDET-Cp) (p=4, 6, 8, 10). Following this methodology, five types of polymers with varying degrees of hydrophobicity were synthesized and no post-synthetic modifications were performed. Statistical incorporation of hydrophobic alkyl chains into the polymer backbone produced polymers with greater hydrophobicity than the parent polyMDET polymer. Importantly, statistical polymers have shown a unique advantage in gene delivery applications, such as enhanced gene transfection and reduced toxicity in the presence of serum compared to common block copolymers (Tan et al., Statistical versus block fluoropolymers in gene delivery, (2018), 6:7230-7238; Ahmed et al., Biomaterials, (2011), 32:5279-5290). ¹H NMR characterization of the synthesized polymers confirmed the presence of characteristic protons in the polymer backbone indicating successful polymerization (FIG. 1C and FIG. 8). The peak at 4.2 ppm confirmed the methylene protons of MDET (—OCH₂—), and the peak around 2.2-2.3 ppm represented the methylene protons of sebacoyl chloride (—OCOCH₂—) at an integration ratio of 1:1, confirming conjugation. Similarly, the incorporation of alkyl chains (Cp) generated an additional peak at 4.0 ppm for the methylene protons (—OCH₂—). The integration ratio of the methylene protons of MDET and Cp was 1:0.3, implying that the percentage of Cp was 30% of the total MDET present in the polymer backbone. To confirm the absence of any unreacted monomers or oligomers, we performed diffusion ordered NMR spectroscopy (DOSY) of polyMDET and polyMDET-Cp polymers (FIG. 9). The DOSY spectrum displays the chemical shifts of NMR resonances against their translational diffusion coefficient, where signals along the same horizontal line belong to the same polymer. Analysis of the DOSY spectrum for individual polymers revealed signals with similar diffusion coefficients consistent with an efficient polymerization and absence of any monomers or oligomers.

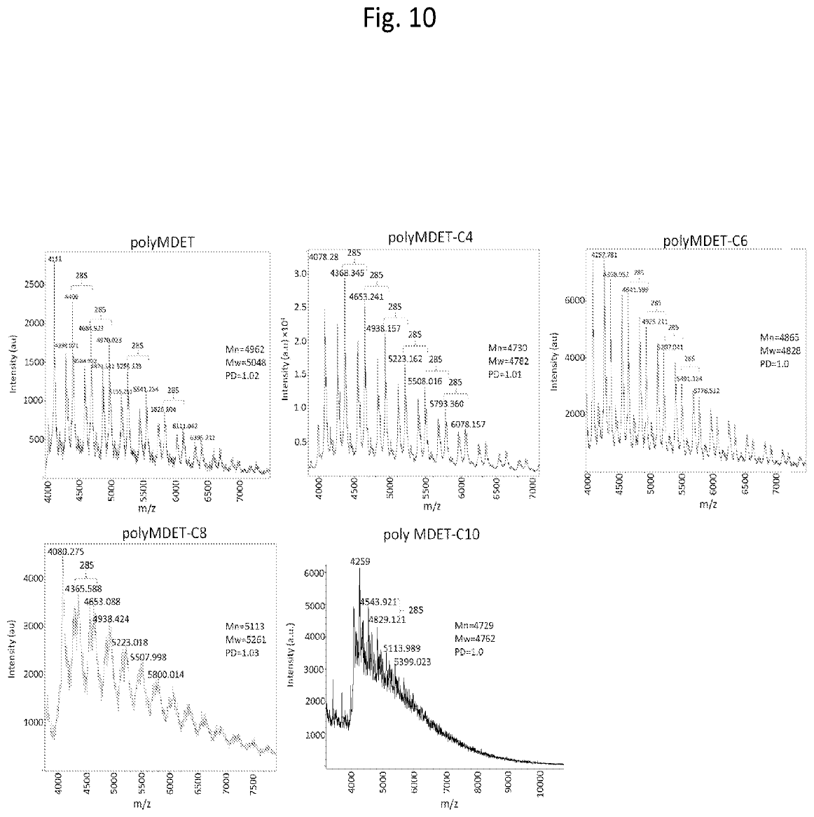

The molecular weight and polydispersity index (PDI) of the polymers was determined by matrix-assisted laser desorption/ionization-time of flight (MALDI-TOF) spectrometry using 2,5 dihydroxybenzoic acid (DHB) as the matrix (FIG. 1D and FIG. 10). The m/z difference (285 Da) between the peaks corresponded to MDET blocks. The number average molecular weight (Mn), weight average molecular weight (Mw), and PDI were calculated using the following equations, where Nᵢ and Mᵢ represent the abundance and mass of the i^{th} oligomer, respectively (Williams et al., Macromolecules, (1997), 30:3781-3787; Lim et al., Pharmaceutical research, (2000), 17:811-6).

$${M}_{n} = \sum {N}_{i} {M}_{i} / \sum {N}_{i}$$

$${M}_{w} = \sum {N}_{i} {M}_{i}^{2} / \sum {N}_{i} {M}_{i}$$

$$PDI = {M}_{w} / {M}_{n}$$

The molecular weight of the polymers was similar and varied between 4-5 kDa demonstrating that the incorporation of different alkyl diols minimally affected the polymerization. While we did not explore the reaction conditions that affect the molecular weight of the polymers synthesized, the same molar ratio for all the starting materials was maintained and the molecular weight of the polymers was found to be similar as shown by MALDI-TOF (FIG. 10) (Eltoukhy et al., Biomaterials, (2012), 33:3594-603).

### Formation of Polyplexes and Their Physicochemical Characterization

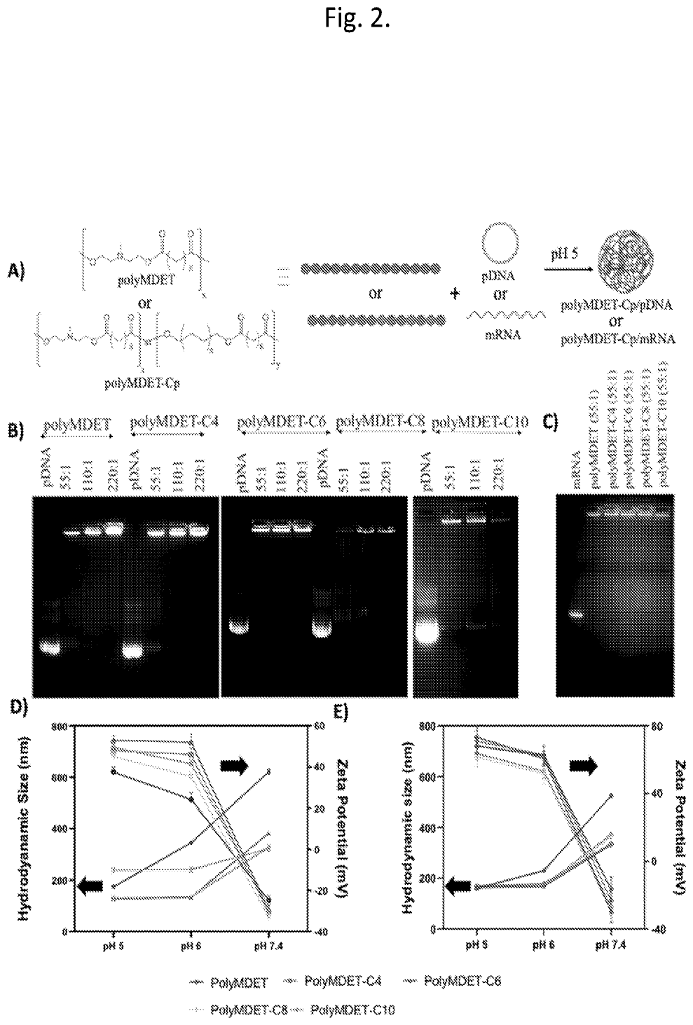

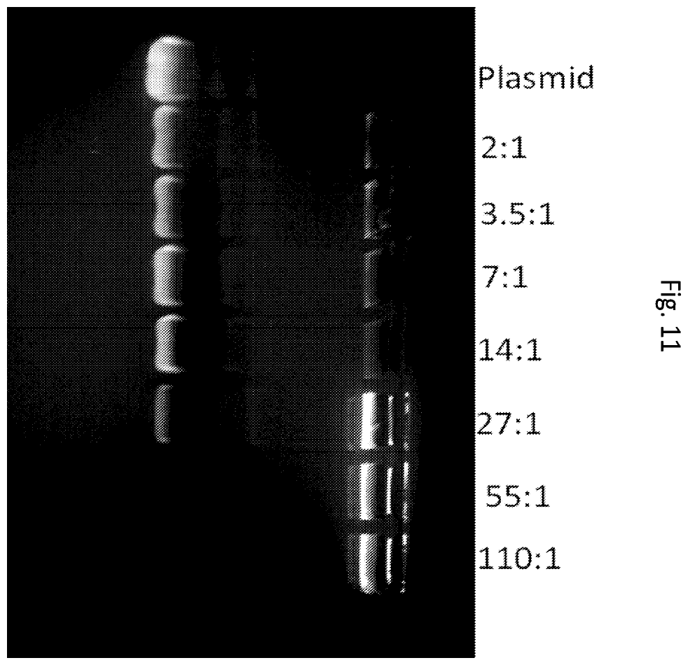

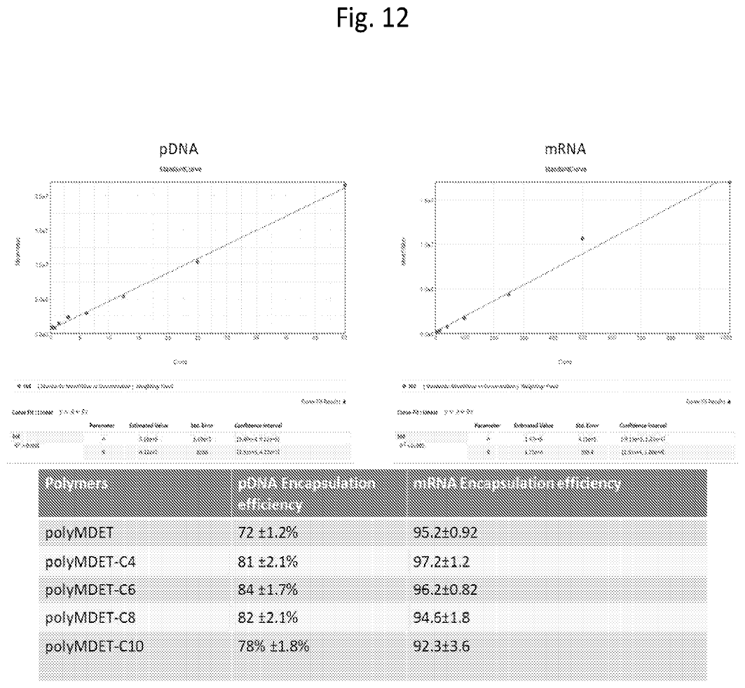

The assembly of macromolecules is governed by the attractive forces operating between them. Cationic polymers interact electrostatically with negatively charged nucleic acids to form condensed structures called polyplexes. Studies have suggested that electrostatic interaction alone is insufficient to offer stability of the polyplexes under physiological conditions due to competition with other electrolytes present (Naeye et al., In vivo disassembly of IV administered siRNA matrix nanoparticles at the renal filtration barrier, (2013), 34:2350-2358). Incorporation of hydrophobic ligands can enhance the cooperative binding with the nucleic acids, which facilitates increases in encapsulation efficiency, reduces the size of polyplexes, and offers improved stability. Further, hydrophobic moieties increase cell membrane interactions, increase dissociation of polyplexes, and release nucleic acid into the cytosol (Sunshine et al., Effects of base polymer hydrophobicity and end-group modification on polymeric gene delivery, (2011), 12:3592-3600). PolyMDET and polyMDET-Cp formed polyplexes with both pDNA (polyMDET/pDNA and polyMDET-Cp/pDNA) and mRNA (polyMDET/mRNA and polyMDET-Cp/mRNA) as shown in FIG. 2A. The stability of polyMDET/pDNA or polyMDET-Cp/pDNA polyplexes at three different weight ratios of polymer to pDNA (55:1, 110:1, and 220:1) was assessed using gel electrophoresis (FIG. 2B). Generally, the polyplexes were stable at 55:1 weight ratio except polyMDET-C10, where a slight band indicating incomplete complexation was observed. PolyMDET-C6 polyplexes prepared at weight ratios lower than 55:1 were unable to stably condense pDNA (FIG. 11). Therefore, we utilized the 110:1 ratio to prepare polyMDET/pDNA and polyMDET-Cp/pDNA polyplexes. PolyMDET and polyMDET-Cp also formed stable polyplexes with mRNA yet a lower weight ratio (55:1) was required (FIG. 2C). We measured the encapsulation efficiency of pDNA and mRNA (FIG. 12), which was found to vary between 72-84% and 92-97%, respectively.

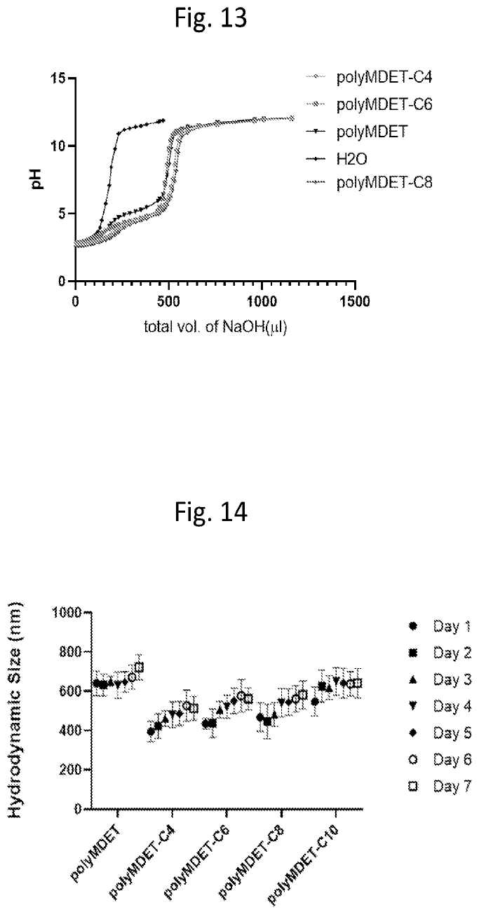

The size and zeta potential of the prepared polyplexes was found to be pH dependent, owing to the ionizable tertiary amines present in the polymer backbones. pH titration curves revealed the charge-altering characteristics of the polymers as shown by pKa values between 4-5 (FIG. 13). Polyplexes were prepared at pH 5 and subsequently dialyzed against DPBS at pH 7.4. During the initial polyplex formation, the zeta potential was greater than 40 mV, however neutralizing the pH resulted in a reversal of the zeta potential to less than-20 mV. Furthermore, the sizes of polyMDET/pDNA, polyMDET-Cp/pDNA, polyMDET/mRNA, and polyMDET-Cp/mR.NA were similar ˜200 nm at pH 5 and increased as a function of pH (FIG. 2D, FIG. 2E, Table 1), although the increase in size was not the same for all the polyplexes.

TABLE 1
Hydrodynamic sizes and PDI of the polyplexes at different pH.

| | Sizes, PDI | | | | | |
|---|---|---|---|---|---|---|
| | pDNA | mRNA | pDNA | mRNA | pDNA | mRNA |
| Polyplexes | pH 5 | pH 5 | pH 6 | pH 6 | pH 7.4 | pH 7.4 |
| polyMDET | 174.4, | 160.2, | 345.3, | 229.7, | 621.4, | 525, |
| | 0.115 | 0.062 | 0.092 | 0.114 | 0.218 | 0.152 |
| polyMDET-C4 | 127.4, | 166.9, | 134.4, | 179.2, | 327.3, | 336, |
| | 0.172 | 0.161 | 0.128 | 0.131 | 0.202 | 0.138 |
| polyMDET-C6 | 127.5, | 165.3, | 133.8, | 168.2, | 382.3, | 332, |
| | 0.092 | 0.216 | 0.112 | 0.192 | 0.231 | 0.151 |
| polyMDET-C8 | 131.8, | 156.9, | 137.5, | 172.4, | 332.1, | 362, |
| | 0.025 | 0.181 | 0.062 | 0.189 | 0.194 | 0.231 |
| polyMDET-C10 | 238.9, | 170.1, | 241.3, | 171.3, | 322.5, | 372.0, |
| | 0.214 | 0.242 | 0.262 | 0.261 | 0.251 | 0.343 |

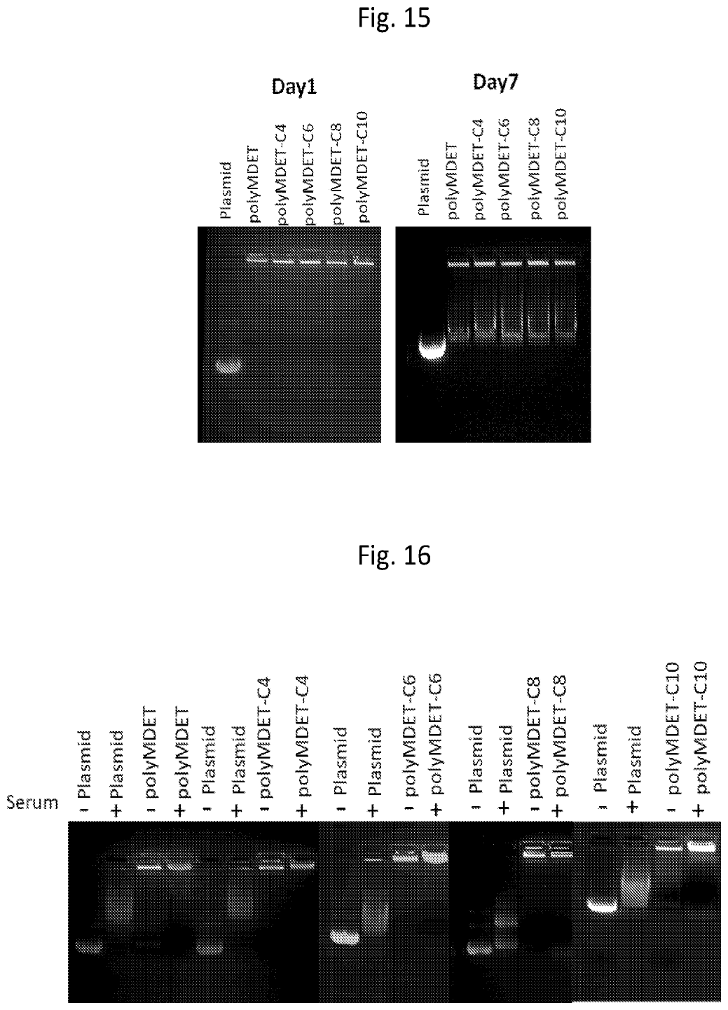

The increased hydrophobicity of polyMDET-Cp versus polyMDET polymers likely contributed to the less significant increase in size at higher pH. Interestingly, the size of polyMDET-C10/pDNA and polyMDET-C10/mRNA was similar at pH 7.4. However, at lower pH, we measured a slight (˜80 nm) difference in size between polyMDET-C10/pDNA and polyMDET-C10/mRNA that could be related to the size of the pDNA compared to mRNA (˜5-fold greater nucleotides). Over the course of 7 days, we observed differential levels of stability for various polyplexes (FIG. 14) and all polyplexes displayed similar size characteristics to their initial formulations for at least 2 days except for polyMDET-C10. Gel electrophoresis also showed that all polyplexes displayed a reduced ability to fully condense pDNA after 7 days (FIG. 15), which can possibly be explained by the degradation of the polymers over time. In fact, another study found that similar MDET containing polyesters that were modified with cholesterol side chains p (MDS-co-CES) lost approximately 54% of their weight in PBS pH 7.4 and 37° C. over the course of 8 weeks (Wang et al., Synthesis and characterization of cationic micelles self-assembled from a biodegradable copolymer for gene delivery, (2007), 8:1028-1037).

### Polyplex Stability in the Presence of Serum and DNase I

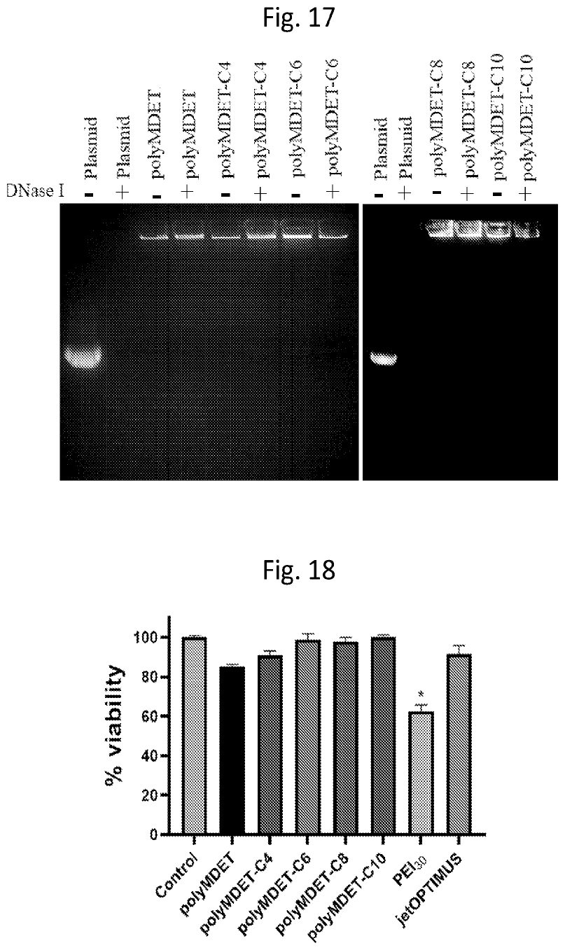

The ability for polyplexes to protect nucleic acid cargoes under physiological conditions was evaluated by incubating polyplexes in a physiologically relevant concentration (55% FBS) of serum or DNase I for 30 min or 1 hour, respectively followed by performing gel electrophoresis (Chakraborty et al., ACS Applied Bio Materials, (2020), 3:6263-6272; Burke et al., Bioconjugate Chemistry, (2008), 19:693-704; Zhao et al., Preparation and efficacy of Newcastle disease virus DNA vaccine encapsulated in PLGA nanoparticles, (2013), 8: e82648; Jacobson et al., ACS Central Science, (2020), 6:2008-2022; Naidu et al., RSC Advances, (2020), 10:2856-2869). Serum incubation revealed partial destabilization of pDNA polyplexes prepared from polyMDET and polyMDET-C10 polyplexes, whereas the other polyplexes remained stable (FIG. 16). FIG. 17 shows that all polyplexes protected DNA from DNase I degradation.

Physicochemical Properties of Polymers Results in Differential In Vitro Transfection of pDNA and mRNA

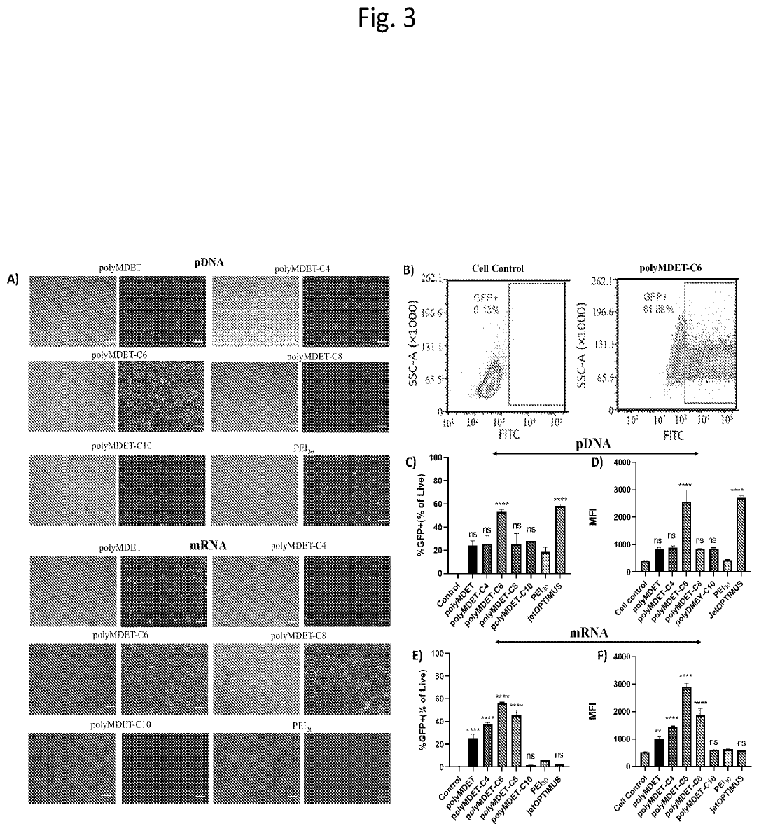

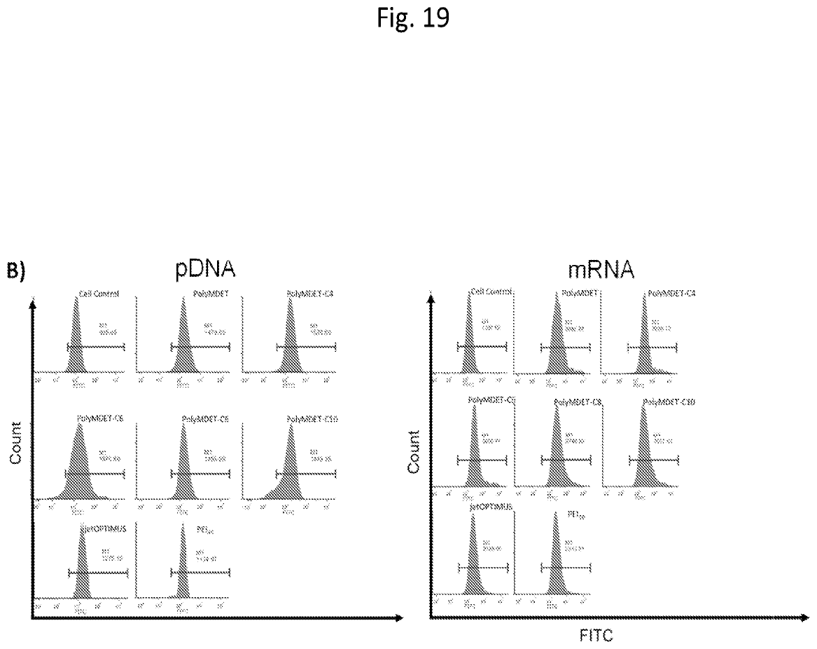

To assess the structure-property relationships between polyplexes and in vitro transfection efficiency, a monocyte/macrophage-like cell line, RAW 264.7 was used. All transfection experiments were performed in the presence of 10% FBS. Fluorescence microscopy and flow cytometry revealed that the transfection efficiency of polyMDET/pDNA polyplexes prepared at 110:1 ratio was similar to PEI (at N/P ratio 30) (FIG. 3A). The N/P ratio of PEI was chosen based on our previous study that showed PEI30 was able to produce higher transfection in macrophages in the presence of serum (Chakraborty et al., ACS Applied Bio Materials, (2020), 3:6263-6272). MTS assay showed that the polyMDET and polyMDET-Cp polyplexes evaluated did not reduce cell viability at the concentrations used, whereas PEI30 displayed a 40% reduction in viability (FIG. 18). The toxicity of PEI is caused by its high positive charge density, which can lead to strong interactions with cell surfaces and subsequent damage (Zintchenko et al., Bioconjugate Chemistry, (2008), 19:1448-1455). The absence of a high positive charge density and pH-dependent charge properties of polyMDET and polyMDET-Cp are likely reasons for the lack of toxicity observed for these polyesters. Hydrophobic modification enhanced the transfection efficiency and reached a maximum for polyMDET-C6/pDNA (3-fold higher compared to PEI30). FIG. 3B demonstrated that 62% of RAW 264.7 cells were GFP⁺compared to untreated cells and the transfection efficiency of polyMDET-C6/pDNA was comparable to another commercially-available transfection reagent, jetOPTIMUS (FIG. 3C, 3D, and FIG. 19).

Transfection of immune cells using mRNA is advantageous compared to pDNA, as it does not require nuclear entry and allows for efficient protein expression in lesser proliferative primary cells. Hence, mRNA transfection has become a mainstay for developing various biomedical applications, including vaccines, protein replacement therapy, and immunotherapies (Islam et al., Nature Biomedical Engineering, (2018), 2:850-864; Kowalski et al., Mol Ther, (2019), 27:710-728; Beck et al., Molecular Cancer, (2021), 20:69). To monitor the mRNA transfection efficiency of the various polyplexes, we utilized GFP reporter mRNA. FIG. 3A shows representative fluorescence microscopy images of the GFP signal. We further quantified the transfection efficiency using flow cytometry (FIG. 3E, 3F, and FIG. 19). Conversely to pDNA transfection, all mRNA-containing polyplexes efficiently transfected RAW264.7 cells except for polyMDET-C10. Notably, the transfection efficiency for the best performing polyMDET-C6/mRNA polyplex was over 10-fold greater than PEI30 and jetOPTIMUS that were used as controls.

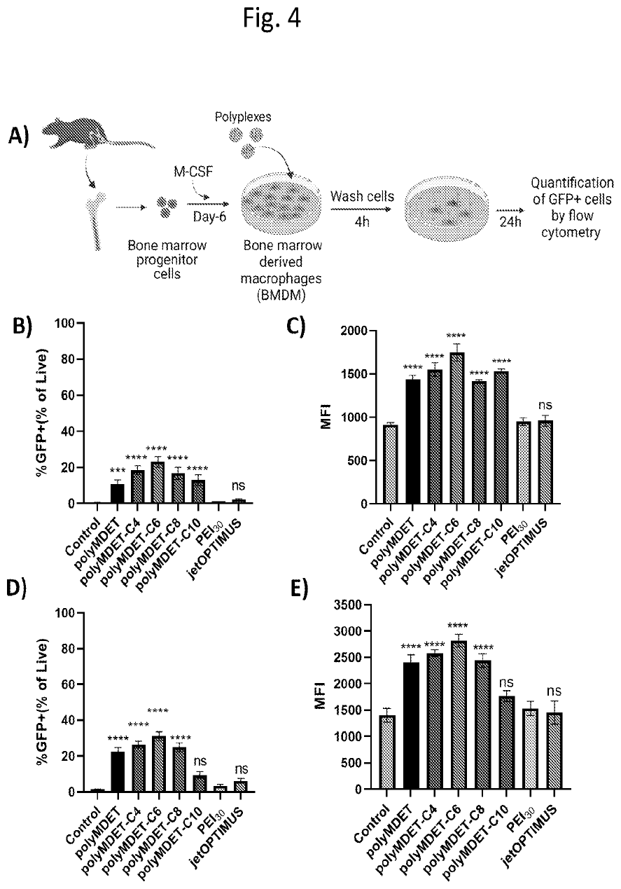

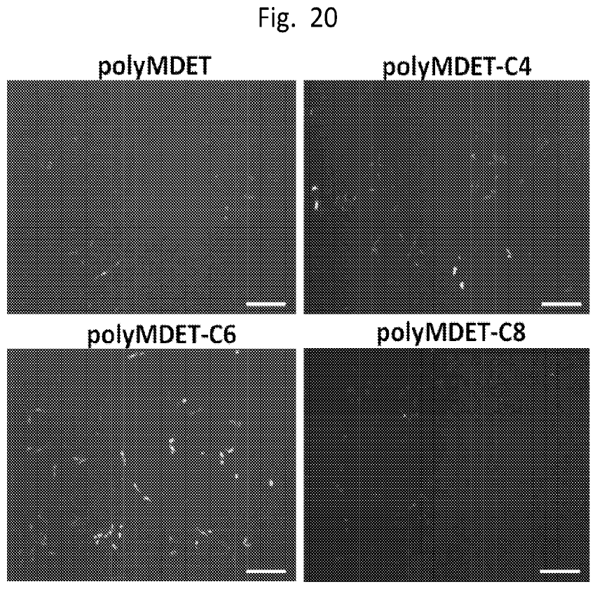

The pDNA and mRNA transfection of polyMDET and polyMDET-Cp polyplexes was next evaluated using primary bone marrow-derived macrophages (BMDM) and compared with PEI30 and jetOPTIMUS as controls (FIG. 4). The transfection efficiency of polyMDET and polyMDET-Cp was significantly higher (3-4 fold) compared to the controls with polyMDET-C6 performing the best followed by polyMDET-C4 and polyMDET-C8, which was further validated using fluorescence microscopy (FIG. 20). Interestingly, there were no differences in the trend for transfection between pDNA and mRNA, although mRNA transfection was 50% more efficient than pDNA (30% versus 20% for polyMDET-C6). Importantly, these results were similar to other efficient PBAE polyplexes that utilized a poly(glutamic acid)-dimannose targeting ligand to enhance the uptake of polyplexes for the transfection of BMDMs (Zhang et al., Nature Communications, (2019), 10:3974).

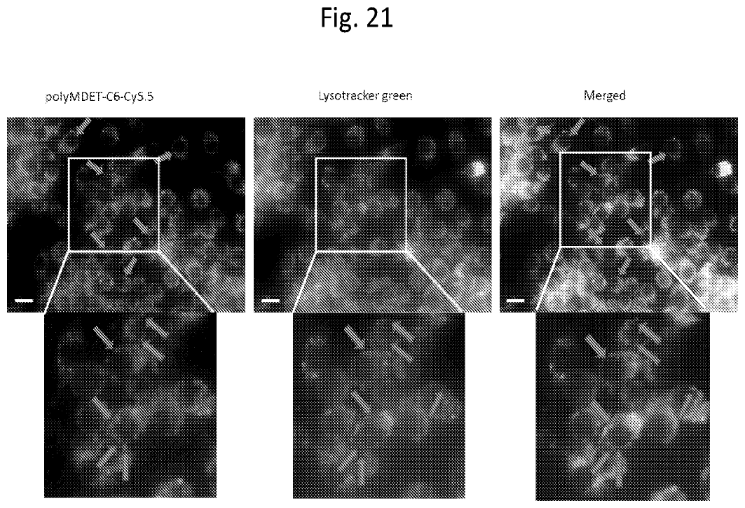

The enhancement of transfection in both RAW 264.7 cells and BMDMs for polyMDET and polyMDET-CP polyplexes without the need to incorporate additional excipients, surface coatings or targeting ligands could be due to multiple reasons. It was reported that the balance between hydrophobicity and cationic charge can significantly affect nucleic acid delivery (Sunshine et al., Effects of base polymer hydrophobicity and end-group modification on polymeric gene delivery, (2011), 12:3592-3600). The hydrophobicity of the polymers can enhance cell membrane interactions, resulting in increased cellular uptake of polyplexes, and improved gene expression (Gao, X., In Gene Therapy for Diseases of the Lung, CRC Press: (2020), pp 99-112; Mohammadinejad et al., Journal of Controlled Release, (2020), 325:249-275; Liu et al., European Journal of Medicinal Chemistry, (2017), 129:1-11). The increased expression of GFP could also be attributed to the efficient endosomal escape of the polymers, where a previous study showed that interplay between the alkyl chain length and the monomer ratio can lead to a significant change in nucleic acid loading, endosomal escape, nucleic acid delivery, and eventually therapeutic outcomes (Foroozandeh et al., Nanoscale Research Letters, (2018), 13:339). Although there are minor differences in the hydrophobic alkyl chains among PolyMDET-C6, PolyMDET-C8, and PolyMDET-C10, it could lead to significant differences in transfection efficiency due to impairment of one or multiple intracellular processes as previously shown (Fortune et al., Journal of drug delivery, (2011), 204058; Sunshine et al., Biomacromolecules, (2011), 12:3592-600; Santos et al., Functionalization of poly(amidoamine) dendrimers with hydrophobic chains for improved gene delivery in mesenchymal stem cells, (2010), 144:55-64). The pKa of the polymers is anticipated to be protonated at endosomal pH (FIG. 13), which could allow for polyplexes to utilize the proton sponge effect to escape endosomes. We confirmed the ability of polyMDET-C6 polyplexes to escape endosomal trafficking by encapsulating Cy5.5 and co-staining using lysotracker for fluorescence imaging (FIG. 21). After 24 h, the observation of polyplexes co-localized and not co-localized with lysotracker staining, in combination with transfection data demonstrating GFP expression in various cell types (FIG. 3 and FIG. 4), supports that the polyplexes were able to escape endosomes. Overall, these results demonstrated that single-component, excipient-free polyMDET and polyMDET-Cp polyplexes were highly efficient in transfecting both pDNA and mRNA in “hard-to-transfect” innate immune cells, including RAW 264.7 cells and primary BMDMs. The hydrophobicity of the polymers was a driving factor for efficient transfection, with moderately hydrophobic polyMDET-C6 offering the highest transfection compared to the more hydrophobic polyMDET-C8 and polyMDET-C10 polyplexes.

### Cellular Uptake Mechanism of Polyplexes

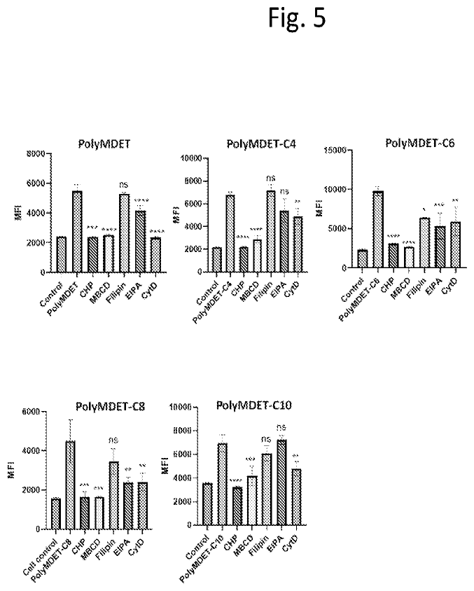

The cellular uptake mechanism of polyplexes was evaluated using RAW 264.7 cells. The intensity of the GFP fluorescence signal from nucleic acid delivery by polyplexes was used to determine the impact of various uptake inhibitors known to alter clathrin-dependent endocytosis (chlorpromazine (CHP)), lipid raft and caveolin-dependent endocytosis (methyl-β-cyclodextrin (MBCD)), caveolin-dependent endocytosis (filipin), micropinocytosis (5-(N-ethyl-N-isopropyl)-amiloride (EIPA), and phagocytosis (cytochalasin D (CytD)) (Islam et al., Nature Biomedical Engineering, (2018), 2:850-864; Hussain et al., J Biol Chem, (2011), 286:309-321). FIG. 5 shows CHP and MBCD treatment completely inhibited polyMDET and polyMDET-Cp induced GFP expression. Filipin treatment reduced GFP expression by polyMDET-C6 by approximately 40%, whereas no significant reductions were observed for other polyplexes. EIPA treatment partially reduced GFP expression from polyMDET, polyMDET-C6, and polyMDET-C8 polyplexes. Lastly, CytD treatment significantly reduced GFP expression induced by all polyplexes with the greatest inhibition noted for polyMDET polyplexes. Taken together, these results indicated that the polyplexes mostly relied on both clathrin- and lipid raft-dependent endocytosis mechanisms to induce gene expression and that blocking one of the pathways was sufficient to eliminate gene expression. All other inhibitors showed variable levels of inhibition that was dependent on the type of polyplex. For example, CytD treatment completely inhibited gene expression induced by polyMDET polyplexes but affected other polyplexes to a lesser, but significant extent. This effect could partially be explained by the differences in the size of polyplexes at pH 7.4. Nanoparticle size plays a crucial role in the endocytosis pathways, which is known to affect their uptake efficiency (Zhang et al., Advanced materials (Deerfield Beach, Fla.), (2009), 21:419-424). Phagocytosis is a pathway by which cells uptake various bacteria, viruses, apoptotic, and necrotic cells. This pathway is known to allow for internalization of particles that are larger in size (0.5-10 μm), which correlated with the DLS results for polyMDET polyplexes being larger than other variants tested (Hashimoto et al., Evidence for phagocytosis of influenza virus-infected, apoptotic cells by neutrophils and macrophages in mice, (2007). 178:2448-2457; Martin et al., Current opinion in microbiology, (2014), 17:17-23).

In Vivo mRNA Delivery and Lung-Selective Transfection

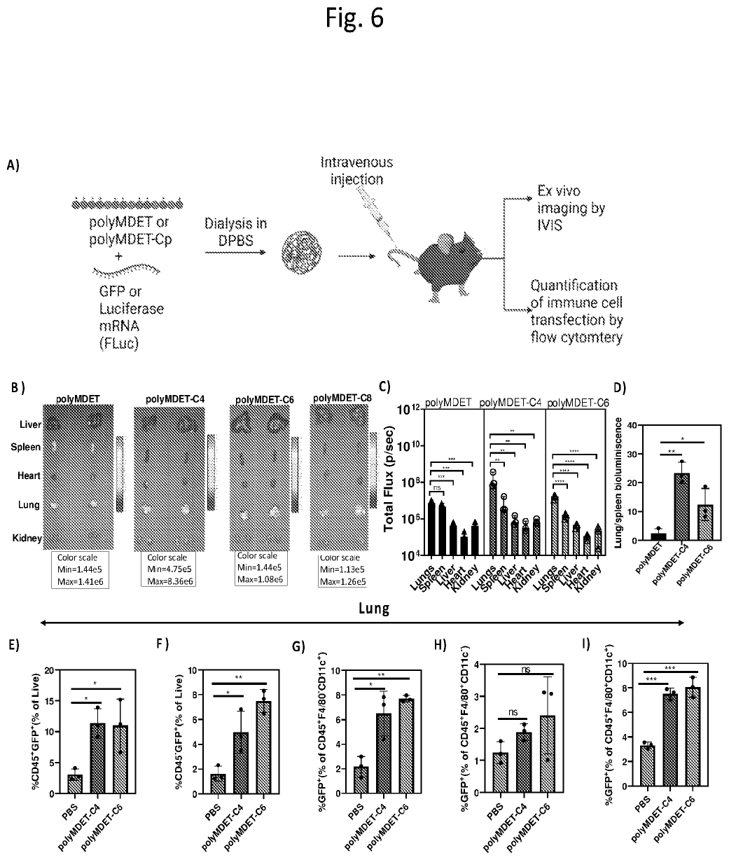

After successful in vitro transfection of pDNA and mRNA, we next explored the efficiency of in vivo transfection with these polyplexes in C57BL/6 mice. Since mRNA was more effective to transfect BMDMs (FIG. 4), we performed these studies using mRNA. Mice were intravenously administered polyMDET/mRNA or polyMDET-Cp/mRNA polyplexes containing 10 μg of luciferase mRNA (FLuc). After 24 hours, the mice were injected with D-luciferin solution intraperitoneally, euthanized, and the luminescence signal from various organs (liver, spleen, heart, lungs, kidneys) was measured using IVIS® (FIG. 6A). A strong luminescence signal was observed in the lungs for certain polyplexes, with the highest signal for polyMDET-C4/mRNA (FIGS. 6B and 6C). PolyMDET showed a strong but equal distribution of protein expression in the lung and spleen with minimal expression in the liver, heart, and kidneys. Interestingly, polyMDET-C4 and polyMDET-C6 displayed a significant increase in the ratio of protein expression in the lung versus the spleen, 23-fold and 12-fold, respectively (FIG. 6D). PolyMDET-C8 polyplexes only showed minor transfection in the spleen. Other organs besides the lung and spleen were minimally transfected.

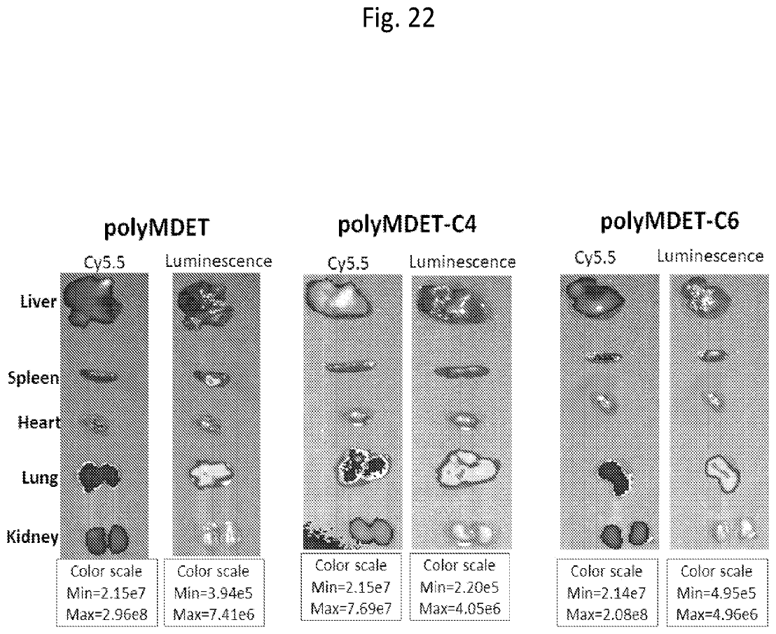

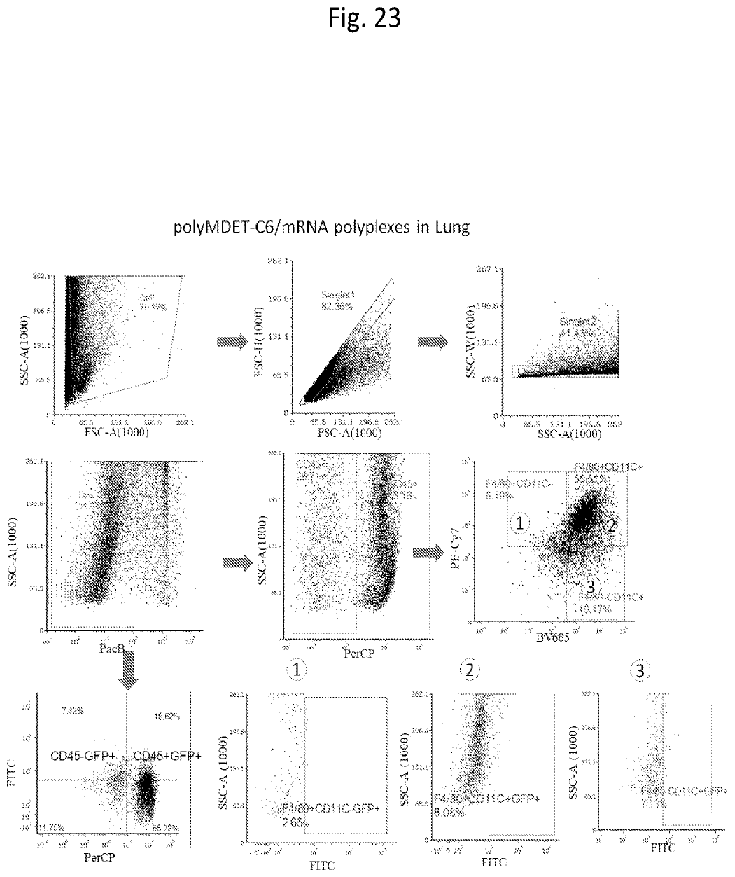

As polyMDET and polyMDET-Cp polyplexes showed slightly different mRNA translation in the lung, we studied the biodistribution of the polyplexes. As shown in FIG. 22, the most significant fluorescence signals were detected in the liver, lung, kidney, and spleen. Previous studies have shown that the pKa and hydrophobicity of polymers affected the organ selectivity of the protein expression (Blanco et al., Nat Biotechnol, (2015), 33:941-951). However, our results differed from previously reported results for LNPs that showed pKa values around 9 led to increased lung selectivity, whereas the pKa for the polymers used in our studies were ≤5 (FIG. 13). Several factors may play a role in the observed differences between our findings and LNPs, including differential uptake and trafficking mechanisms (FIG. 5), differential protein corona fingerprints, and other physicochemical properties including size and zeta potential (Shaw et al., Biomaterials Science, (2022), 10:2540-2549; Pearson et al., Frontiers in Immunology, (2014), 2 (108)). Taken together, these results support that the physicochemical properties of polyMDET-C4/mRNA and polyMDET-C6/mRNA polyplexes played a major role in determining the lung selectivity of FLuc expression.

### In Vivo Immune Cell Transfection

To quantify the immune cell populations transfected in the lung and spleen, we prepared GFP mRNA-containing polyMDET-C4 and polyMDET-C6 polyplexes. Polyplexes containing 10 μg of mRNA were intravenously administered via the tail vein into C57BL/6 mice and the resulting GFP signal in the lungs and spleen was measured using flow cytometry after 24 hours. The gating strategy used for analysis is shown in FIG. S16. We enumerated GFP expression in a variety of cell populations (FIG. 6E-I), such as CD45+ (lymphocytes), CD45⁻ (non-lymphocytes, which includes endothelial and epithelial cells, among others), CD45⁺F4/80⁺CD11c⁺ (alveolar macrophages), CD45⁺F4/80⁺CD11c⁻ (interstitial macrophages), and CD45⁺F4/80⁻CD11c⁺ (dendritic cells) (Abernathy et al., Journal of Thoracic Oncology, (2015), 10:1703-1712; Zaynagetdinov et al., American journal of respiratory cell and molecular biology, (2013), 49:180-9; Misharin et al., American journal of respiratory cell and molecular biology, (2013), 49:503-10.; Bedoret et al., The Journal of Clinical Investigation, (2009), 119:3723-3738). PolyMDET-C4 and polyMDET-C6 polyplexes transfected approximately 10-11% of lymphocytes, 5-8% of non-lymphocytes, 6-8% of dendritic cells, 2-3% of interstitial macrophages and 7-8% of alveolar macrophages.

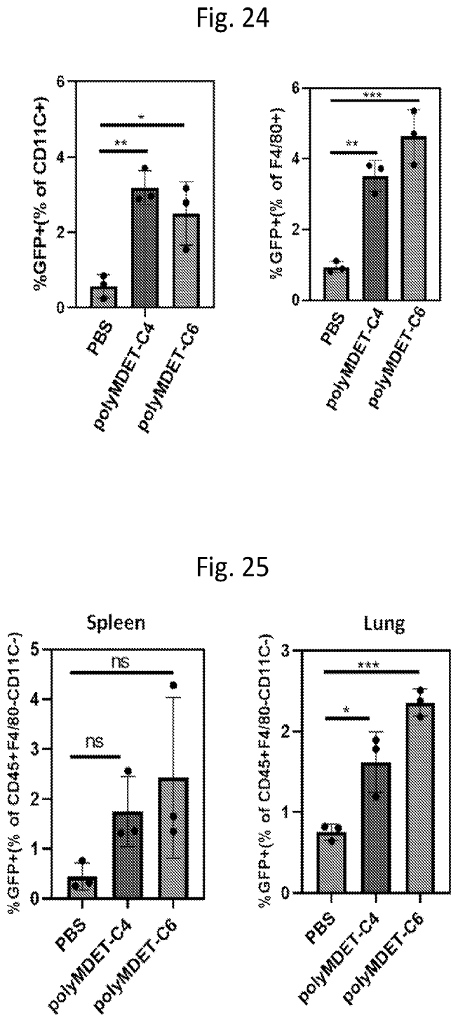

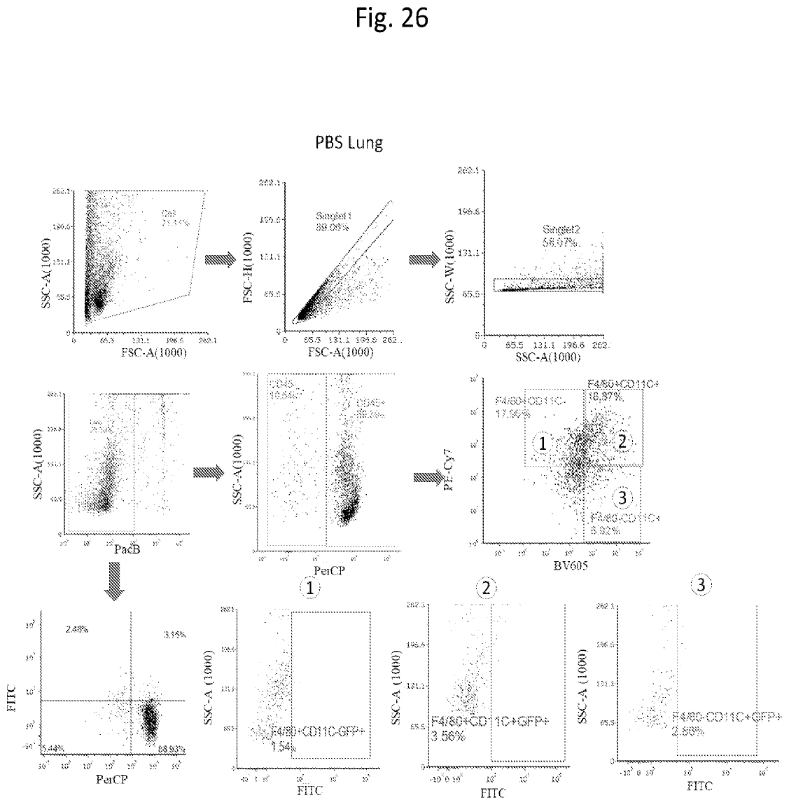

Total cell transfection in spleen was less than the lung for both polyplexes evaluated, which correlated with the IVIS® images that showed a 10-fold lower luciferase signal in the spleen for both polyplexes evaluated. Lymphocyte transfection in the spleen was also lower than the lungs with 2.5-3% of CD45⁺CD11c⁺dendritic cells and 3.5-4.6% of CD45⁺F4/80⁻macrophages being found GFP⁺ (FIG. 24). The reduced transfection within the spleen may be related to distinct differences in cell populations between the spleen and lungs, mainly the significant presence of B and T cells. This corresponded with our in vitro studies evaluating these polyplexes for T cell transfection (Jurkat cells), where no transfection was observed (data not shown). The percentage of CD45⁺F4/80⁻CD11c⁻GFP⁺ cells was also calculated in both the spleen and lungs (FIG. 25), which would include immune cells besides macrophages and dendritic cells, which could include monocytes, neutrophils, B cells, and T cells, among others. The spleen did not show any significant difference compared to PBS, which corroborated the findings that few lymphocytes were transfected. Moreover, 1.5-2.5% of CD45⁺F4/80⁻CD11c⁻ cells were GFP⁺in the lungs. These studies demonstrated that appreciable levels of mRNA transfection can be achieved using polyMDET-C4 and polyMDET-C6 polyplexes in an organ-selective manner to reach “hard-to-transfect” innate immune cells in vivo. Future studies aimed to further distinguish lymphocyte and non-lymphocyte populations within the lung and spleen may better classify differences between polyMDET-C4 and polyMDET-C6 polyplexes.

### Analysis of Organ Tissue Histology and Plasma Cytokines Following Polyplex Treatment

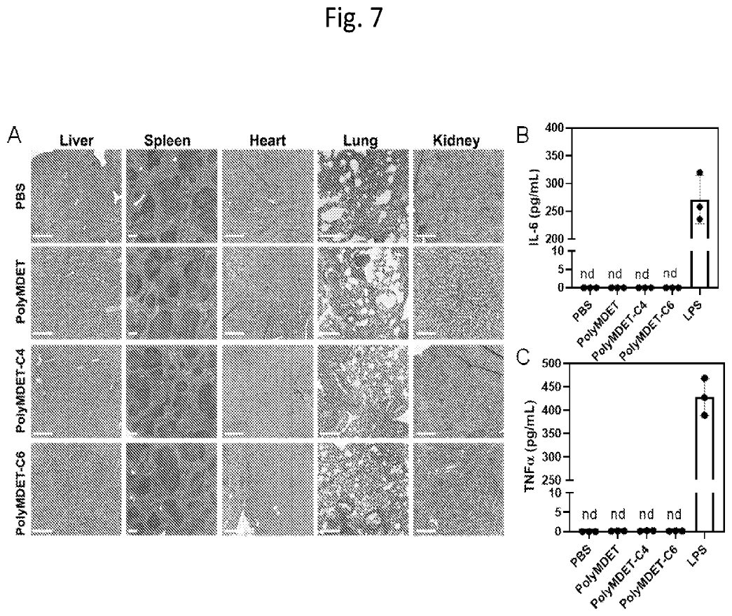

To assess the biocompatibility of polyMDET and polyMDET-Cp polyplexes used in these studies, we performed histological examination and plasma cytokine analyses. Mice were injected intravenously with polyplexes containing 10 μg mRNA via tail vein injection. After 24 h several major organs (liver, spleen, heart, lung, and kidney) were collected, fixed, and stained using hematoxylin and eosin (H&E). This timepoint was chosen for evaluation as it corresponded to the timepoint used for in vivo transfection experiments in FIG. 6. Examination of the tissue sections did not indicate induction of inflammation nor alterations from normal tissue architecture after polyplex treatment (FIG. 7A). LNP administration has been shown to induce proinflammatory responses, owing to the use of ionizable lipids, and strategies have been developed to reduce this effect (Zhang et al., J. Biomed. Mater. Res. A., (2022), 110:1101-1108; Ndeupen et al., iScience, (2021), 24:103479). We analyzed the plasma proinflammatory cytokine levels using an enzyme-linked immunosorbent assay (ELISA) following polyplex treatment. Interleukin-6 (IL-6) and tumor necrosis factor α (TNFa) and were not significantly increased compared to the PBS control, which demonstrated the lack of proinflammatory responses generated by the polyplexes (FIG. 7B). Taken together, these results support the biocompatibility, anti-inflammatory properties, and safety of the polyMDET and polyMDET-Cp polyplexes for in vivo use. Future toxicology studies will be useful to comprehensively evaluate the dose- and time-dependent responses following polyplex administration in a therapeutically-relevant model.

### CONCLUSION

Designing polymer-based carriers for efficient DNA/mRNA transfection is critical for developing nucleic acid therapeutics. Here, we synthesized a set of biodegradable hydrophobic ionizable polyesters (polyMDET and polyMDET-Cp) using a one-pot synthetic methodology by incorporating variable length alkyl chains into the polymer backbone to create a single-component and excipient-free, non-viral polyplex for pDNA and mRNA delivery. Polyplexes were highly efficient to transfect pDNA and mRNA in RAW 264.7 macrophages and primary BMDMs. The hydrophobicity of the polyMDET and polyMDET-Cp was a conducive factor for efficient transfection, with the moderately hydrophobic and best performing polyMDET-C4 and polyMDET-C6 polyplexes achieving over 10-fold higher transfection than PEI30 and jetOPTIMUS in BMDMs with negligible cytotoxicity. Uptake mechanism studies using polyMDET and polyMDET-Cp polyplexes revealed that transfection was eliminated by clathrin, and lipid raft inhibitor treatment, whereas the extent of inhibition due to other inhibitors was polyplex-dependent. Intravenous administration of polyplexes identified a hydrophobicity-driven shift in the lung: spleen protein expression ratio with all protein expression being extrahepatic. Protein expression in the lungs and spleen by the more hydrophilic polyMDET polyplexes was similar, whereas polyMDET-C4 and polyMDET-C6 polyplexes displayed a lung-selective tropism for mRNA transfection of 23- and 12-fold, respectively. Flow cytometry analysis of the lungs showed 6-8% of dendritic cells and 7-8% of alveolar macrophages were transfected with mRNA, while other lymphocytes were marginally transfected (˜2%) demonstrating significant opportunity to modulate innate immune cell responses.

The preferential lung transfection illustrates the importance of polyplex physicochemical properties to modulate biodistribution and immune cell uptake profiles. We envision that altering the size and surface chemistry of the polyplexes may aid in achieving transfection in hepatic or other extrahepatic tissues as previously described (Cheng et al., Nature nanotechnology, (2020), 15:313-320). Importantly, our data support non-inflammatory mRNA delivery to the lung using polyMDET and polyMDET-Cp polyplexes, which offers significant potential to develop tolerogenic mRNA vaccines for the treatment of autoimmunity⁶⁵ or allergy based on several studies utilizing polymeric nanoparticles for induction of antigen (Ag)-specific immune tolerance (Krienke et al., Science (New York, N.Y.), (2021), 371:145-153; Saito et al., Science advances, (2020), 6 (42); Casey et al., Biotechnology & Bioengineering, (2022), doi: 10.1002/bit.28252; Freitag et al., Gastroenterology, (2020), 158:1667-1681.e12; Casey et al., Biomaterials, (2022), 283, 121457. Alternatively, the incorporation of nucleic acid adjuvants targeting Toll-like receptor signaling or the cGas/STING pathway may provide the opportunity to develop targeted cancer vaccines (Miao et al., Nat Biotechnol, (2019), 37:1174-1185; Van Herck et al., Advanced drug delivery reviews, (2021), 179:114020).

### Experimental Section

Materials: N-methyldiethanolamine (MDET), 1,4-butanediol, 1,6-hexanediol, 1,8-octanediol, 1,10-decanediol, sebacoyl chloride, pyridine, anhydrous dichloromethane (DCM, >99.8%), methanol (>99.9%), diethyl ether, chloropromazine hydrochloride (CHP), 5-[N-ethyl-N-isopropyl]-amiloride (EIPA), methyl-β-cyclodextrin (MBCD), cytochalasin D (CytD), and filipin were purchased from MilliporeSigma (St. Louis; MO). Dimethylsulfoxide-d6 and chloroform-d were purchased from Cambridge Isotope Labs Inc (Tewksbury, MA). Maxiprep Endotoxin-free kit was purchased from Qiagen (Germantown, MD). Unless otherwise noted, any additional reagents were purchased from MilliporeSigma.

RAW 264.7 Cell Culture: RAW 264.7 cells were cultured in Dulbecco's Modified Eagle Medium (DMEM) (MilliporeSigma; St. Louis, MO) supplemented with 10% heat-inactivated fetal bovine serum (FBS) (VWR; Radnor, PA) and 1% penicillin/streptomycin (P/S) (Invitrogen Corporation; Carlsbad, CA) at 37° C. and 5% CO₂ atmosphere.

Bone Marrow-Derived Macrophage (BMDM) Cell Culture: Bone marrow from the tibia and femurs of C57BL/6 mice was harvested to obtain a primary population of macrophages as previously described by our group (Truong et al., The AAPS Journal, (2021), 24:6). Cell media consisted of RPMI 1640 supplemented with L-glutamine (Life Technologies, Carlsbad, CA), penicillin (100 units/mL), streptomycin (100 μg/mL), and 10% heat-inactivated fetal bovine serum (FBS) (Invitrogen Corporation, Carlsbad, CA) at 37° C. and 5% CO₂. The media was further supplemented with 20% L929 (ATCC, Manassas, VA) cell-conditioned medium (containing M-CSF). BMDMs were differentiated for 8 days, with cell conditioned media changes on days 3 and 6. Versene (Invivogen, San Diego, CA) was used for BMDM cell lifting. Cell count and viability were obtained using a trypan blue exclusion dye and the EVE™ automated cell counter (NanoEntek, Waltham, MA).

GFP Plasmid Preparation: DH5α Escherichia coli competent cells (Invitrogen Corporation; Carlsbad, CA) were transformed with eGFP (GFP) pDNA (courtesy of the National Center for Toxicological Research, FDA, Jefferson, AR), which encoded for kanamycin resistance. Transformed cells were expanded in an overnight liquid LB culture at 37° C. under vigorous shaking, lysed, and purified using a Qiagen Maxiprep endotoxin-free kit. The concentration of pDNA was verified using a SpectraMax iD3 microplate reader and a SpectraDrop microvolume microplate (Molecular Diagnostics; San Jose, CA) by measuring absorbance at 260 and 280 nm and agarose gel electrophoresis. The pDNA was stored at −20° C. until further use.

Polymer Synthesis: The synthesis of diethanolamine-based ionizable polyester (polyMDET): The diethanolamine-based ionizable polyester (polyMDET) was synthesized by one-pot condensation polymerization using N-methyldiethanolamine (MDET) and sebacoyl chloride in the presence of pyridine as an HCl scavenger. Initially, 595 μL of MDET (5 mmol) was dissolved in 5 mL DCM and added to a 50 mL round bottom flask. A total of 808.79 μL (10 mmol) of pyridine was added and stirred under an Ar atmosphere for 15 minutes. Next, 1 mL of sebacoyl chloride (5 mmol) was dissolved in 5 mL of DCM and slowly added to the reaction mixture for 30 minutes. After complete addition, the reaction was further carried out overnight (16-18 hours). The white precipitate, pyridine hydrochloride, was removed by centrifugation, and the supernatant containing DCM was removed using a rotary evaporator. Next, the resulting solid product was dissolved in a minimum amount of methanol. Finally, the polymer was purified by precipitation into diethyl ether to remove unreacted monomers and oligomers. This process was repeated for a total of three times. The product was dried for two days at room temperature, and ¹H NMR spectra were obtained. Similarly, other polymers were also synthesized, maintaining the same polyester backbone, but integrating hydrophobic aliphatic chains (C4, C6, C8, C10) via mixing respective alkyl diols into the reaction mixture. The respective polymers were termed polyMDET-Cp, with p=the number of carbon atoms present in the diols used during the reaction. For example, polyMDET-C10 was prepared by mixing 1 mmol of 1,10-decanediol with 5 mmol of MDET and 10 mmol of pyridine in 5 mL DCM. As described earlier, the reaction was carried out by adding 6 mmol of sebacoyl chloride dropwise to the reaction mixture under Ar atmosphere.

Polymer Characterization: The polymers were characterized by ¹H NMR after dissolving the samples in DMSO-d6 or CDCl₃. ¹H NMR spectra was acquired using a 400 MHz Varian spectrometer. The diffusion measurement was carried out by observing the attenuation of the ¹H NMR signals during a pulsed field gradient experiment using a 500 MHz Varian spectrometer in DMSO-d6. The molecular weight and polydispersity index (PDI) of the polymers was determined using MALDI-TOF mass spectrometry. The polymer and 2,5 dihydroxybenzoic acid (DHB) matrix were dissolved in methanol at 1 mg/mL and mixed. The mixture was dried on a metal sample plate and placed in the high vacuum source chamber for measurement by analyzing “time of flight” of the produced sample ions. The most probable peak was calculated from the spectra, which was reported as the molecular weight of the polymer.

pH Titration of Polymers: 1 mg/mL of each polymer was dissolved in DMSO and acidified with HCl solution (PH˜2). This solution was titrated with 0.1 M NaOH by adding dropwise to the solution and final pH was measured using a pH meter. pKa was calculated from the inflection point of the obtained titration curve, which was identified as the pH at half of the neutralization point. Polyplex Preparation and Characterization: PolyMDET/pDNA or polyMDET-Cp/pDNA polyplexes were prepared by mixing polyMDET or polyMDET-Cp polymers with pDNA at different weight ratios (55:1, 110:1, or 220:1). Briefly, polymer solutions were prepared at a concentration of 50 mg/mL in DMSO. Next, different amounts of polymer solutions were diluted in 50 μL acetate buffer (25 mM pH 5), and 2 μg pDNA encoding GFP was added to the solution and incubated for 10 minutes at room temperature. Similarly, polyMDET/mRNA or polyMDET-Cp/mRNA polyplexes were also prepared by mixing polyMDET and polyMDET-Cp polymers with 1 μg GFP encoded mR.NA (CleanCap® EGFP mRNA. TriLink Biotechnologies, San Diego, CA) at 55:1 weight ratio in the presence of 25 mM acetate buffer (pH 5). The encapsulation efficiency of pDNA and mRNA polyplexes was determined using 4′,6-diamidino-2-phenylindole (DAPI) or RediPlate 96 RiboGreen RNA quantitation kit (Invitrogen Corporation; Carlsbad, CA), respectively (Zintchenko et al., Bioconjugate Chemistry, (2008), 19:1448-1455; Lei et al., A Combination Therapy Using Electrical Stimulation and Adaptive, Conductive Hydrogels Loaded with Self-Assembled Nanogels Incorporating Short Interfering RNA Promotes the Repair of Diabetic Chronic Wounds, (2022), 2201425). The polyplexes were prepared as described above, centrifuged, and the supernatant was collected for analysis. DAPI binds with the double stranded DNA and gives rise to 20-fold enhancement of fluorescence intensity. The RiboGreen fluorescent dye binds to mRNA and produces a fluorescent signal that is proportional to mRNA content. The fluorescence signal was measured using a SpectraMax iD3 fluorescence plate reader and the pDNA and mRNA content was determined by comparing to respective standard curves.

The polyplexes were characterized by dynamic light scattering (DLS) and zeta potential analysis. Fresh polyplex solutions were prepared with 2 μg of GFP plasmid or mRNA in three different buffer solutions (pH 5, pH 6, and pH 7.4). One milliliter of each sample was added into a disposable cuvette, and the size was measured using a Zetasizer Nano ZSP (Malvern Instruments Inc., Westborough, MA). Fifteen runs were performed in triplicate for each sample. Subsequently, samples were transferred to a folded capillary cell (Malvern), and zeta potential measurements were performed in triplicate for each sample using the Zetasizer Nano ZSP.

The stability of polyplexes was assessed by gel electrophoresis before or after incubating with 55% FBS, a physiologically relevant serum concentration, for 30-60 min. The success of a nucleic acid delivery platform relies on protecting the genetic material from enzymatic degradation, for example. DNase I present in serum, extracellular matrices, and mucosal surfaces (Apostolov et al., Radiation Research, (2009), 172:481-492). Therefore, the ability of the polyplexes to protect DNA was investigated using agarose gel electrophoresis. First, polyplexes were prepared, as described earlier, with polyMDET or polyMDET-Cp, and incubated with 20/K unit DNase I for 1 hour at room temperature. After that, the integrity of DNA was assessed by agarose gel electrophoresis (150 V, 20-40 min).

In Vitro Transfection of pDNA in RAW 264.7 Macrophages and Primary BMDMs: PolyMDET/pDNA or polyMDET-Cp/pDNA polyplexes were prepared at a weight ratio of 110:1 containing 2 μg of pDNA encoding GFP. RAW 264.7 cells were seeded in a 24 well plate at 2×10⁵ density and treated with polyplexes (2 μg pDNA/220 μg polyMDET or polyMDET-Cp) for 4 hours in serum-containing media, followed by washing to remove the excess complex prior to overnight incubation in complete media. The excess complex was removed by washing with DPBS and replaced with complete DMEM. Similarly, primary BMDMs were incubated with polyplexes for 4 hours in serum-containing RPMI media. After that, the cells were washed with DPBS and incubated with fresh complete RPMI media for 24 hours. GFP expression was visualized using a revolve fluorescence microscope (ECHO, San Diego, CA) 24 hours post-transfection. Cationic polymers PEI and jetOPTIMUS were used as positive controls for pDNA transfection. PEI/pDNA polyplexes (PEI30) were prepared at N/P ratio 30, as PEI30 showed significantly higher transfection in serum-containing media according to our previous study (Chakraborty et al., ACS Applied Bio Materials, (2020), 3:6263-6272). Transfection with jetOPTIMUS was achieved by following the manufacturer's protocol.

In Vitro Transfection of mRNA in RAW 264.7 Macrophages and Primary Bone Marrow-Derived Macrophages: PolyMDET/mRNA or polyMDET-Cp/mRNA polyplexes were prepared by mixing polymer solution with GFP mRNA at a 110:1 weight ratio (1 μg mRNA/110 μg polyMDET or polyMDET-Cp). Next, RAW 264.7 macrophages or primary BMDMs were treated with the polyplexes (24 well plate, 2×10⁵ density) and incubated for 4 hours in serum-containing media. After that, the cells were washed with DPBS and further incubated with fresh complete media for 24 hours. GFP expression was visualized using a revolve fluorescence microscope at 24 hours post-transfection. PEI30 and jetOPTIMUS were also used as positive controls for mRNA transfection.

Endosomal Escape of Polyplexes: To examine the endosomal escape, particles were prepared by mixing PolyMDET-C6 with Cy5.5 dye at a 110:1 weight ratio by mixing at pH 5 (acetate buffer). The particles were dialyzed against DPBS buffer (pH 7.4) for 1 hour to remove free Cy5.5. Next, RAW 264.7 cells were incubated with the particles for 3 h followed by washing and incubation with fresh complete DMEM media for 24 h. The cells were then treated with 100 nM of lysotracker green for 15 minutes and washed with DPBS buffer (pH 7.4). The cells were imaged using a revolve fluorescence microscope to visualize the colocalization of polyplexes with lysotracker.

Flow Cytometry: RAW 264.7 or primary BMDMs were seeded in a 24 well plate at a density of 2×10⁵ cells/well. Next, cells were transfected with polyplexes made with different polymers and GFP encoded pDNA/mRNA in serum-containing media for 4 hours. Subsequently, cells were washed and incubated for another 24 hours in fresh serum-containing media. After 24 hours, cells were collected using a cell scraper, followed by centrifugation at 500×g for 5 min, and resuspended in fresh flow cytometry buffer (DPBS, 5% FBS and 2% EDTA). For analysis of live cells only, DAPI was used as an exclusion dye to determine cell viability. Data were collected using an LSR II (Becton-Dickinson, San Jose, CA) flow cytometer and analyzed by FCS Express 7 (De Novo Software, Pasadena, CA). Transfection efficiency was measured as the percentage of live cells, which were GFP+ compared to non-transfected controls.

Uptake Mechanism Study using Endocytosis Inhibitors: RAW 264.7 cells were seeded in a 24 well plate and cultured for 24 hours in complete DMEM media at a 2×10⁵ cell density/well. The cells were preincubated in presence or absence of chlorpromazine (CHP) (50 μM), methyl-β-cyclodextrin (MBCD) (10 mM), Filipin (1 μg/μL), (5-[N-ethyl-N-isopropyl] amiloride) (50 μM) (EIPA), Cytochalasin D (CytD) (4 μM) for 30 min. The cells were then treated with polyplexes polyMDET/pDNA or polyMDET-Cp/pDNA for another 3 hours. The cells were subjected to flow cytometry analysis after being washed with DPBS and incubated with fresh DMEM for 24 hours. The cellular uptake of the polyplexes was evaluated by measuring the mean fluorescence intensity (MFI) of GFP signals.

Cell Viability: The cytotoxicity of polyplexes was evaluated using an MTS assay (Abcam; Cambridge, MA). RAW 264.7 cells were seeded in a 24 well plate at a density of 2×10⁵ cells/well overnight prior to treatment with polyMDET/pDNA and polyMDET-Cp/pDNA polyplexes for 4 hours in serum-containing media. Following the incubation, cells were washed and incubated with fresh complete DMEM media and incubated for 24 hours. Next, 50 μL of MTS solution was added to each well and incubated for an additional 3 hours. The optical density (OD) of the solution was measured using a SpectraMax iD3 microplate reader at 570 nm, and the percentage of cell viability was measured as the ratio of OD at 570 nm and compared to untreated control.

In Vivo mRNA Transfection: Female C57BL/6 (6-8 weeks old) were purchased from the Jackson Laboratories. The mice were housed under specific pathogen-free conditions in the School of Medicine, University of Maryland, Baltimore animal facilities. All animal procedures were performed according to the guidelines and protocols of the University of Maryland, Baltimore Animal Care and use committee and approved by the Institutional Animal Care and use Committee (IACUC; protocol #0721010). Polyplexes were prepared in acetate buffer (25 mM; pH 5) by mixing different polymers (50 mg/mL) and FLuc mRNA (1 mg/mL) (CleanCap® FLuc mRNA (5moU), TriLink Biotechnologies, San Diego, CA) at 110:1 weight ratio. After the preparation, the polyplexes were dialyzed against DPBS buffer (pH 7.4) for 30-60 minutes to remove DMSO and acetate buffer. All the polymers were stable after dialysis except polyMDET-C10/mRNA. The polyplexes were administered to mice via tail vein injection (10 μg mRNA/injection). After 24 hours, the mice were injected with D-luciferin (300 μL, 15 mg/mL) intraperitoneally. After 15 minutes, the mice were euthanized, and various organs were collected (liver, spleen, heart, lung, kidneys) and imaged using the Xenogen IVIS® Spectrum Imaging System (Alameda, CA).

Biodistribution Study of mRNA Polyplexes In Vivo: The polyplexes were prepared by mixing the polymers and Fluc mRNA at a 110:1 weight ratio. The polyplexes were then mixed with 10 μg of Cy5.5 (1 mg/mL) and incubated for 60 minutes at room temperature. The polyplexes were then dialyzed against DPBS buffer (pH 7.4) for 30 minutes to remove excess Cy5.5 dye. Next, the polyplexes were injected in mice intravenously by tail vein injection. After 24 hrs, the localization and transfection of polyplexes were evaluated by IVIS® imaging as described above. Cy5.5 fluorescence indicates the organ trafficking of polyplexes whereas luminescence signal indicates the luciferase expression.

Flow Cytometry For In Vivo Studies: For the evaluation of immune cell transfection, female C57BL/6 (6-8 weeks old) were intravenously injected with GFP mRNA polyMDET-C4/mRNA and polyMDET-C6/mRNA polyplexes (10 μg mRNA/injection). After 24 hours, the mice were euthanized, and lung and spleen were collected. Single cell suspensions were prepared using a standardized protocol. First, tissue samples were placed in a petri dish and injected with Liberase (Liberase TM for lungs and Liberase TL for spleen; MilliporeSigma) followed by incubation for 10 minutes at 37° C. After that, cells were isolated by mashing the lung and spleen through a 70 μm cell strainer (Thermo Fisher) and treated ACK lysis buffer (Thermo Fisher). Cells were then pelleted by centrifuging at 500×g for 5 minutes, followed by resuspension in DPBS supplemented with 10% FBS. Cell staining was conducted according to BioLegend protocols. All antibodies were purchased from BioLegend (San Diego, CA). Flow cytometric data were collected using a BD LSR II flow cytometer. FcR blocking was performed with anti-CD16/32 (clone 98) antibody prior to staining with extracellular antibodies: PerCP anti-mouse CD45 (clone 30-F11), PE/Cy7 anti-mouse F4/80 (clone BM8), and BV605 anti-mouse CD11c (clone N418). Viability was assessed using DAPI. Data analysis was performed using FCS Express 7 (De Novo, Glendale, CA).

Histopathological Analysis: Organs were harvested after intravenous administration of DPBS or mRNA polyplexes prepared with PolyMDET, PolyMDET-C4, and PolyMDET-C6. After 24 h, the mice were euthanized and the liver, spleen, heart, lungs, and kidneys were collected and fixed in 4% paraformaldehyde in DPBS (pH 7.4) for 24 h for sectioning. The organ tissues were dehydrated and embedded in paraffin before being sectioned and stained with haematoxylin and eosin (H&E) and observation using the revolve microscope under brightfield.

Detection of Cytokines by Enzyme Linked Immunosorbent Assay (ELISA): Polyplexes containing Fluc mRNA were prepared as described above and administered via tail vein injection. After 24 h, the blood was collected by cardiac puncture and stored in EDTA-coated tubes. Within 30 minutes of blood collection, the blood was centrifuged at 1000×g to separate the cellular fraction from the plasma. The plasma was then utilized to assess the proinflammatory cytokine levels for tumor necrosis factor-alpha (TNFa) and interleukin-6 (IL-6) using ELISA following the manufacturer's protocols (BioLegend, San Diego, CA).

1. An ionizable polyester having the following structure: 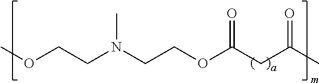
wherein a is an integer in a range of from 5 to 12; and
wherein m is in a range of from about 11 to about 190.

2. The ionizable polyester of claim 1, wherein the polyester has the following structure: 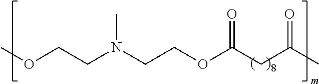
wherein m is in a range of from about 14 to about 160.

3. The ionizable polyester of claim 2, wherein the polyester is prepared by condensation of N-methyldiethanolamine (MDET) and sebacoyl chloride.

4. An ionizable polyester having the following structure: 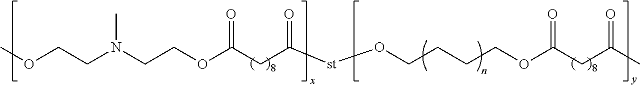
wherein n is 1, 2, 3, 4, 5 or 6;
wherein x is in a range of from about 10 to about 130;
wherein y is in a range of from about 3 to about 40; and
wherein “st” signifies that the polyester includes a statistical distribution of the two bracketed primary units.

5. The ionizable polyester of claim 4, wherein the polyester is prepared by condensation of N-methyldiethanolamine (MDET), sebacoyl chloride and a 1,p-alkanediol, wherein p is [(n*2)+2], and wherein the condensation is performed as a one pot synthesis.

6. The ionizable polyester of claim 4, where a hydrophobicity of the polyester increases with increasing n.

7. The ionizable polyester of claim 4, wherein y to x ratio is in a range of from about 0.25 to about 0.35.

8. The ionizable polyester of claim 1, wherein the polyester has high serum and enzymatic stability when administered to a subject.

9. The ionizable polyester of claim 1, wherein the polyester is biodegradable.

10. The ionizable polyester of claim 1, wherein the polyester exhibits low toxicity when administered to a subject.

11. The ionizable polyester of claim 1, wherein the molecular weight of the polyester is in a range of from about 4000 daltons to about 4500 daltons.

12. The ionizable polyester of claim 1 wherein the pKa for the polymer is ≤5.

13. A method of preparing the ionizable polyester of claim 1, comprising: providing q moles of N-methyldiethanolamine (MDET) dissolved in a first nonaqueous solvent; mixing 2q moles of a proton scavenger with the MDET; stirring under an inert atmosphere for about 15 minutes; adding q moles of sebacoyl chloride dissolved in a second nonaqueous solvent to the mixture of MDET with the proton scavenger over about 30 minutes to provide a reaction mixture; continuing to stir the reaction mixture for 12 hours under an inert atmosphere; forming a solid hydrochloride salt of the proton scavenger during the adding and continuing steps; removing the solid hydrochloride salt from the reaction mixture; and evaporating the solvent(s) to provide a crude polyester product.

14. The method of claim 13, further comprising:
dissolving the crude polyester product in a minimum amount of methanol to form a methanol solution; precipitating a purified polyester product by adding the methanol solution to diethyl ether; isolating the purified polyester product by filtration; and drying the purified polyester product for two days at room temperature.

15. The method of claim 13, wherein the first nonaqueous solvent and the second nonaqueous solvent are dichloromethane.

16. The method of claim 13, wherein the proton scavenger is pyridine.

17. The method of claim 13, wherein the inert atmosphere is argon.

18. The method of claim 13, wherein the removing step is accomplished by centrifugation.

19. The method of claim 13, wherein the evaporating step is accomplished with a rotary evaporator.

20. The method of claim 13, wherein the concentration of MDET in the first nonaqueous solvent in the providing step is about 1.0M.

21.-62. (canceled)
